(19) United States
(12) Patent Application Publication
(10) Pub. No.: US 2024/0058432 A1
(43) Pub. Date: Feb. 22, 2024

(54) HUMAN PAPILLOMAVIRUS TYPE 58 CHIMERIC PROTEIN AND USE THEREOF

(71) Applicant: Institute of Basic Medical Sciences, Chinese Academy of Medical Sciences, Beijing (CN)

(72) Inventors: Xuemei XU, Beijing (CN); Zhirong WANG, Beijing (CN); Mingrao MA, Beijing (CN); Ting ZHANG, Beijing (CN)

(73) Assignee: Institute of Basic Medical Sciences, Chinese Academy of Medical Sciences, Beijing (CN)

(21) Appl. No.: 18/259,711

(22) PCT Filed: Sep. 26, 2021
(86) PCT No.: PCT/CN2021/120608

§ 371 Date: Jun. 28, 2023

(30) Foreign Application Priority Data
Jan. 4, 2021 (CN) 202110003706.3

(51) Int. Cl. A61K 39/12; C07K 14/005; A61P 31/20

(52) U.S. Cl. CPC A61K 39/12; C07K 14/005; A61P 31/20; A61K 2039/5258

(57) ABSTRACT

Provided are an HPV chimeric protein and a use thereof. The HPV chimeric protein of the present invention comprises an HPV58 L1 protein or a mutant thereof and a polypeptide derived from a HPV16 L2 protein and inserted into a surface region of the HPV58 L1 protein or the mutant thereof, or consists of the polypeptide, wherein an amino acid sequence of the HPV58 L1 protein is as shown in SEQ ID No. 1 and an amino acid sequence of the HPV16 L2 protein is as shown in SEQ ID No. 2.

### FIELD OF THE INVENTION

The present invention relates to the field of biotechnology. Specifically, the present invention relates to a human papillomavirus chimeric protein, and a pentamer or a virus-like particle formed thereby, as well as use of the human papillomavirus chimeric protein, the pentamer or the virus-like particle formed by the human papillomavirus chimeric protein in the preparation of a vaccine for the prevention of papillomavirus infection and infection-induced diseases.

### BACKGROUND OF THE INVENTION

Human papillomaviruses (HPVs) are a class of envelope-free small DNA viruses that infect epithelial tissues. More than 200 types have been identified and classified into α, β, γ, μ, and η genera according to the amino acid homology of the main coat protein L1. They are also classified into mucosa types and skin types according to the different sites of infection. Mucosa type HPVs mainly infect the genitourinary, perianal and oropharyngeal mucosa and skin. They all belong to the α genus, and are classified into oncogenic HPVs with transforming activity and low-risk HPVs (LR-HPVs) that induce benign hyperplasia. Oncogenic HPVs include 12 common high-risk types (including HPV16, -18, -31, -33, -35, -39, -45, -51, -52, -56, -58, -59, etc.), 1 probable high-risk type (HPV68), and more than 10 possible high-risk types (HPV26, -30, -34, -53, -66, -67, -69, -70, -73, -82, -85, etc.). Studies have found that all oncogenic HPV-positive cancerous tissues show specific E6*I mRNA expression, decreased expression of tumor suppressor gene Rb/P53 and cyclin CD1, and increased expression of p16INK 4a, indicating that the oncogenic risk from infection with any type of oncogenic HPV is the same. There are about 12 types of low-risk HPVs (HPV6, -7, -11, -13, -32, -40, -42, -43, -44, -54, -74, -91, etc.), of which HPV types 6 and -11 induce a total of 90% of perianal and genital condyloma acuminata and most of the recurrent respiratory papillomas. Skin-type HPVs mainly infect skin tissues other than the above sites, some of which (HPV2, -27, -57) induce skin verrucous hyperplasia, and other types (HPV5, -8, -38, etc.) are associated with the occurrence of squamous cell carcinoma and basal cell carcinoma of skin.

Malignant tumors that have been identified as related to oncogenic HPV infection are cervical cancer, vaginal cancer, vulval cancer, penile cancer, anal and perianal cancer, oropharyngeal cancer, tonsil cancer and oral cancer, among which cervical cancer is the most harmful. Cervical cancer is the third highest incidence of malignant tumor in women in the world, with an annual incidence of about 527,000, including 285,000 in Asia and 75,000 in China. The 12 common high-risk types of HPV induce a total of 95.2%-96.5% of cervical cancer, and the rest more than 10 probable and possible high-risk types induce a total of about 3.29% of cervical cancer. HPV type 16 is a prevalent high-risk type worldwide, with the highest detection rate in HPV-related tumors such as cervical cancer, perianal cancer, penile cancer, vulval cancer, etc. and in precancerous lesions. The detection rates of HPV16 and -18 in cervical cancer worldwide are 50-60% and −20%, respectively. The detection rate of HPV58 and -52 in cervical cancer in southern China is second only to HPV16 or HPV16/-18. In Asia, the overall detection rate of the high-risk type HPV58 is next to HPV16 and HPV18 types, and the detection rates in cervical cancer, high-grade cervical endometrial neoplasia and low-grade cervical endometrial neoplasia are relatively high, being 7.3%, 15.5% and 10.8%, respectively, all ranking third. In Central and South America, the detection rates of HPV58 in cervical intraepithelial neoplasia III (CIN3) specimens are as high as 11.6% and 11.0%, respectively, ranking second. In Mexico, the detection rate of HPV58 in cervical precancerous lesion specimens is even greater than or equal to that of HPV16. Therefore, the prevalence of HPV58 infection in these economically underdeveloped areas is relatively high, and the public health burden and economic burden caused by infection-related diseases are relatively heavy. The 12 common high-risk types of HPV induce a total of 95.2%-96.5% of cervical cancer, and the rest more than 10 probable and possible high-risk types induce a total of about 3.29% of cervical cancer.

HPV L1 virus-like particles (L1VLPs) mainly induce type-specific neutralizing antibodies and protective responses, and the protective range of vaccines consisting of L1 virus-like particles can only be expanded by increasing the types of L1VLPs. The three HPV vaccines on the market are all L1VLP vaccines, namely the divalent vaccine by GSK (Cervarix, HPV16/-18), the tetravalent vaccine (Gardasil, HPV6/-11/-16/-18) and the nine-valent vaccine (Gardasil-9, HPV6/-11/-16/-18/-31/-33/-45/-52/-58) by Merck, of which the nine-valent vaccine with the widest protection range only covers limited 7 high-risk types and 2 low-risk types (HPV6/-11), and cannot prevent the skin types. In addition, the protection range of L1VLP vaccines cannot be expanded by unlimitedly increasing the types of L1VLPs. Therefore, L1VLP vaccines can hardly meet the requirements of prevention of HPV infection-related diseases.

The secondary capsid protein L2 of HPV has no immune activity in its native state, but the N-terminal polypeptide of L2 can induce cross-neutralizing antibodies and cross-protection responses, although the immunogenicity is weak, the titer of the induced antibody is low, and the types cross-neutralized by monotypic L2 antiserum are limited. At present, a variety of conserved epitope peptides that can induce neutralizing antibodies have only been found in 16 L2N, in which aa. 17-38 is its main neutralizing epitope region, and the monoclonal antibody RG-1 recognizing this region cross-neutralizes the most types. Therefore, this region is also called RG-1 epitope peptide, in which aa. 21-31 is the core sequence of its neutralizing epitope. The homologous region of aa. 21-31 is always retained in studies related to RG-1 epitope peptides, regardless of the length of the sequence.

The RG-1 types used in vaccine research include HPV type 4 RG-1, HPV type 6 RG-1, HPV type 16 RG-1, HPV type 17 RG-1, HPV type 31 RG-1, HPV type 33 RG-1, HPV type 45 RG-1, HPV type 51 RG-1, HPV type 58 RG-1, etc., and the employed means include VLP surface display, bacterial protein surface display (bacterial thioredededin Trx, flagellar protein, cholera toxin mutant CRM197), targeted IgyR modified antibody and tandem fusion of multiple types of L2 polypeptides containing the RG-1 epitope. However, research results have shown that a variety of RG-1 epitope peptide-related vaccines have poor activity results. For example, the three types of 16cVLPs surface displaying HPV type 4 RG-1, HPV type 6 RG-1 or HPV type 17 RG-1 induced very low titer of HPV16 neutralizing antibody, and the titer of cross-neutralization was not detected. The 18cVLP displaying HPV type 45 RG-1 induced very low titer of HPV18 neutralizing antibody (only 1/100 of 18L1VLP), and only cross-neutralized the oncogenic types HPV45, -70 and -39 with very low titers, the highest was only 100 [B. Huber et al., PLoS One 2015, 10(3):e0120152]. The antiserum of Trx fusion protein surface displaying HPV type 51 RG1 had a narrow cross-neutralization range, and the highest titer of cross-neutralizing antibody was only 500.

In another aspect, the 16RG1-cVLP reported by Schellenbacher as well as the 31RG1-cVLP, 33RG1-cVLP and 58RG1-cVLP reported by the inventor's research group had better immunoactivity, and the titer of HPV16 neutralizing antibody induced by the backbone type VLP was as high as 10⁵ (comparable to that induced by 16L1VLP), and the corresponding RG-1 epitope-induced L2-dependent cross-neutralizing antibodies had a wide neutralization range and relatively high titers (the highest could be as high as 6400) [C. Schellenbacher et al., The Journal of investigative dermatology 2013, 133 (12): 2706-13; X. Chen et al., Oncotarget 2017, 8(38): 63333-63344; X. Chen et al., Human Vaccines & Immunotherapeutics 2018, 14(8):2025-2033; PCT/CN2017/075402].

The above data suggest that different types of RG-1 epitope peptides have different immunogenicity. The immunogenicity of 58RG-1 and 6RG-1 was compared for the first time in a publication, and it was found that 58RG-1 epitope peptide antiserum cross-neutralized more types (13 types) and had higher titers (the highest was as high as 3200), while 6RG-1 epitope peptide antiserum neutralized fewer types (9 types) and had very low titers (the highest was only 100) [X. Chen et al., Oncotarget 2017, 8(38):63333-63344]. This indicated that although the RG-1 epitope peptide region was strongly conserved among different types, the different types of RG-1 had different immunogenicity. Therefore, the immune activity of a chimeric protein vaccine constructed with aa.17-36 homologous polypeptide selected from any type of L2 cannot be expected.

It was worth noting that the HPV16 cVLP vaccine study reported by Schellenbacher and Wang showed that when inserting a 16RG-1 epitope peptide into the surface region of a 16L1VLP vector, due to the difference in the flanking sequence, the insertion site and insertion method of the type 16RG-1 core epitope peptide sequence, the immune activity of the multiple different type 16RG1-cVLPs obtained was significantly different. Among them, the cVLP with insertion of 16RG-1 into the DE loop region of 16L1 had the best activity, and the cVLP with insertion of 16RG-1 core sequence into the h4 region of 16L1 had the poorest activity. In addition, Chen and Boxus both reported the cVLP of 33RG-1, but different vectors were used, being HPV16L1VLP and 18L1VLP, respectively. Both reports chose the DE loop as the insertion site, while the insertion region differed by 1 amino acid, and the epitope peptide length differed by 2 amino acids, the two resulting 33RG1-cVLPs induced very significantly different activity of the 33RG-1-dependent cross-neutralizing antibodies. 33RG1-cVLP antiserum could cross-neutralize at least 12 types (2 types of which had a titer >1000), while 33RG1-18cVLP antiserum only cross-neutralized 7 types, of which 6 types had a much lower neutralization titer (4 types of which had a titer <100) than that of the 33RG1-16cVLP antiserum. Therefore, the above data show that even if RG-1 epitopes with strong immunogenicity are selected, the immune activity and expression amount of cVLPs obtained by construction are different due to different vectors, different insertion sites, different flanking sequences, and different insertion methods. Therefore, the available research data show that the type of RG-1 polypeptide and its length (difference in epitope flanking sequences), the type of L1VLP vector and its insertion site and insertion method (direct insertion, substitution insertion and introduction of amino acids such as linkers into the insertion site region) have an effect on the expression level, assembly ability and immune activity of the formed RG1-L1 chimeric protein, and this effect is unpredictable.

Currently, there is a need to develop a vaccine based on chimeric proteins of HPV58 L1 and HPV L2 that can produce high-titer neutralizing antibodies against more types of HPV viruses, which can both maintain or enhance the neutralizing epitope of HPV58 L1 and provide cross-protection against more HPV types.

### SUMMARY OF THE INVENTION

A variety of 16RG-1 epitope peptides of different lengths are selected in the present invention for the study of HPV type 58 chimeric pentamer or cVLP. The results show that the HPV58 chimeric pentamer or cVLP obtained by the present invention has very strong immunogenicity, the level of neutralizing antibodies induced against the vector type HPV58 is comparable to that of 58L1VLP, and can induce broad-spectrum neutralizing antibodies against multiple types of HPVs from different genera/subgenera. In view of this, the object of the present invention is to provide a human papillomavirus chimeric protein for the preparation of a vaccine for the prevention of papillomavirus infection and infection-induced diseases. The inventors have unexpectedly found that the insertion of a polypeptide derived from HPV type 16 L2 protein into the surface region of wild-type HPV type 58 L1 protein or mutants thereof can improve the immunogenicity of HPV type 16 L2 protein polypeptide. The obtained chimeric protein can be expressed at a high level in an E. coli or insect cell expression system. The chimeric protein can be assembled into VLP or chimeric pentamer, and can induce a broad-spectrum protective immune response against multiple types of HPVs from different genera/subgenera.

Based on the above object, the present invention provides a human papillomavirus chimeric protein, the backbone of which is a HPV type 58 L1 protein or a mutant of the HPV type 58 L1 protein, and the backbone is embedded with at least one polypeptide derived from a HPV type 16 L2 protein.

That is, in a first aspect, the present invention provides a human papillomavirus chimeric protein comprising or consisting of a HPV type 58 L1 protein or a mutant of the HPV type 58 L1 protein, and a polypeptide from a HPV type 16 L2 protein inserted into the surface region of the HPV type 58 L1 protein or the mutant of the HPV type 58 L1 protein, wherein the amino acid sequence of the HPV type 58 L1 protein is as shown in SEQ ID No. 1, and the amino acid sequence of the HPV type 16 L2 protein is as shown in SEQ ID No. 2.

In a preferred embodiment of the human papillomavirus chimeric protein according to the present invention, the polypeptide from the HPV type 16 L2 protein is selected from any continuous fragment of 8-33 amino acids in the region of aa. 1-50 of the HPV type 16 L2 protein as shown in SEQ ID No. 2. Further preferably, the polypeptide from the HPV type 16 L2 protein is a HPV type 16 L2 protein RG-1 epitope peptide or a mutant epitope peptide thereof.

In a preferred embodiment of the human papillomavirus chimeric protein according to the present invention, the amino acid sequence of the polypeptide from the HPV type 16 L2 protein is as shown in SEQ ID No. 3, SEQ ID No. 4, SEQ ID No. 5 or SEQ ID No. 6.

In a further preferred embodiment, the polypeptide from the HPV type 16 L2 protein is a polypeptide obtained by extending or truncating 1-7 amino acids at the N-terminus and/or extending or truncating 1-7 amino acids at the C-terminus of the amino acid sequence as shown in SEQ ID No. 3.

In a further preferred embodiment, the polypeptide from the HPV type 16 L2 protein can also be a polypeptide with greater than 60%, preferably greater than 70%, greater than 80%, greater than 90%, and even more preferably greater than 95% of sequence identity with the amino acid sequence as shown in SEQ ID No. 3.

In a preferred embodiment of the present invention, the chimeric protein backbone involved in the present invention is selected from the HPV type 58 L1 protein (e.g., the sequence as shown in CAX48979.1 in the NCBI database, consistent with SEQ ID No. 1) or mutants of the HPV type 58 L1 protein. The HPV type 58 L1 protein backbone can be from, but not limited to, L1 proteins of HPV58 variant strains such as AFS33402.1, ADK78323.1, AMY16498.1, ACJ13512.1, ADK78590.1, ADK78685.1 in the NCBI database. Preferably, the amino acid sequence of the HPV type 58 L1 protein is as shown in SEQ ID No. 1.

In a preferred embodiment of the human papillomavirus chimeric protein according to the present invention, compared with the HPV type 58 L1 protein as shown in SEQ ID No. 1, the mutant of the HPV type 58 L1 protein according to the present invention comprises any one or more selected from the group consisting of deletion mutation, C-terminus truncation mutation and substitution mutation, wherein:
the deletion mutation is a deletion of amino acids at positions 2-4 at the N-terminus;
the C-terminus truncation mutation is a 25-amino acid truncation at the C-terminus;
the substitution mutations are any group selected from the following groups i) to iii):
i) 476G, 481G, 492G, 493G, 497G, 478S, 487S, 494S, 498S, 480A and 495A;
ii) 474G, 476G, 481G, 492G, 493G, 497G, 478S, 487S, 494S, 498S, 480A and 495A; and
iii) 476G, 481G, 492G, 493G, 497G, 478S, 494S, 498S, 480A and 495A.

In the representation of the substitution mutation used herein, the number in the middle represents the amino acid position compared to the control sequence (e.g., the amino acid sequence as shown in SEQ ID No. 1), the letter preceding the number (if any) represents the amino acid residue before mutation, and the letter succeeding the number represents the amino acid residue after mutation.

Alternatively, the mutant of the HPV type 58 L1 protein is a protein obtained by truncating 0-8 amino acids at the N-terminus and/or truncating 0-25 amino acids at the C-terminus of the HPV type 58 L1 protein.

Alternatively, the mutant of the HPV type 58 L1 protein is a mutant with a truncation of amino acids at positions 2-4 at the N-terminus and/or a 25-amino acid truncation at the C-terminus of the amino acid sequence of the HPV type 58 L1 protein.

Alternatively, the mutant of the HPV type 58 L1 protein is a mutant (CS1) with a truncation of amino acids at positions 2-4 at the N-terminus of the amino acid sequence of the HPV type 58 L1 protein and substitutions of amino acids 476, 481, 492, 493, 497 of the HPV type 58 L1 protein to glycine (G), amino acids 478, 487, 494, 498 to serine (S), and amino acids 480 and 495 to alanine (A).

Alternatively, the mutant of the HPV type 58 L1 protein is a mutant (CS2) with a truncation of amino acids at positions 2-4 at the N-terminus of the amino acid sequence of the HPV type 58 L1 protein and substitutions of amino acids 474, 476, 481, 492, 493, 497 of the HPV type 58 L1 protein to glycine (G), amino acids 478, 487, 494, 498 to serine (S), and amino acids 480 and 495 to alanine (A).

Alternatively, the mutant of the HPV type 58 L1 protein is a mutant (CS3) with a truncation of amino acids at positions 2-4 at the N-terminus of the amino acid sequence of the HPV type 58 L1 protein and substitutions of amino acids 476, 481, 492, 493, 497 of the HPV type 58 L1 protein to glycine (G), amino acids 478, 494, 498 to serine (S), and amino acids 480 and 495 to alanine (A).

Alternatively, the polypeptide from the HPV type 16 L2 protein is inserted into the surface region of the HPV type 58 L1 protein or the mutant of the HPV type 58 L1 protein, preferably inserted into the DE loop of the HPV type 58 L1 protein or the mutant of the HPV type 58 L1 protein, more preferably inserted between amino acids 136 and 137, or between amino acids 431 and 432 of the HPV type 58 L1 protein or the mutant of the HPV type 58 L1 protein by direct insertion, or inserted into the region of amino acids 429 to 432, or the region of amino acids 426 to 429, or the region of amino acids 412 to 426 of the HPV type 58 L1 protein or the mutant of the HPV type 58 L1 protein by non-isometric substitution.

As used herein, the term “direct insertion” refers to the insertion of a selected peptide fragment between two adjacent amino acids. For example, direct insertion between amino acids 136 and 137 of SEQ ID No. 1 refers to the direct insertion of the selected peptide fragment between amino acids 136 and 137 of SEQ ID No. 1.

As used herein, the term “non-isometric substitution” refers to the insertion of a selected peptide fragment into the specified amino acid region after deleting the sequence of the specified amino acid region. For example, non-isometric substitution of the region of amino acids 429 to 432 of SEQ ID No. 1 refers to the insertion of the selected peptide fragment between amino acids 429 and 432 of SEQ ID No. 1 after deleting amino acids 430-431 of SEQ ID No. 1.

Alternatively, in an embodiment of direct insertion or non-isometric substitution, the polypeptide derived from the HPV type 16 L2 protein comprises a linker of 1 to 3 amino acid residues in length at its N-terminus and/or C-terminus.

Alternatively, the linker consists of any combination of amino acids selected from the group consisting of glycine (G), serine (S), alanine (A) and proline (P). Preferably, the linker at the N-terminus consists of G (glycine) P (proline), and the linker at the C-terminus consists of P (proline).

Alternatively, in an embodiment of direct insertion, the amino acid sequence of the polypeptide from the HPV type 16 L2 protein is SEQ ID No. 6, the insertion site is between amino acids 136 and 137 of the HPV type 58 L1 protein or the mutant of the HPV type 58 L1 protein with a 25-amino acid truncation at the C-terminus, and the amino acid sequence of the obtained papillomavirus chimeric protein is as shown in SEQ ID No. 7 or SEQ ID No. 8.

Alternatively, in an embodiment of insertion by non-isometric substitution, the amino acid sequence of the polypeptide from the HPV type 16 L2 protein is SEQ ID No. 6, the insertion site is the region of amino acids 429-432 of the HPV type 58 L1 protein or the mutant of the HPV type 58 L1 protein with a 25-amino acid truncation at the C-terminus, after deleting the region of amino acids 430-431 of the HPV type 58 L1 protein or the mutant of the HPV type 58 L1 protein, the polypeptide as shown in SEQ ID No. 6 is inserted between amino acids 429 and 432, and the amino acid sequence of the obtained papillomavirus chimeric protein is as shown in SEQ ID No. 9 or SEQ ID No. 10.

Alternatively, in an embodiment of insertion by non-isometric substitution, the amino acid sequence of the polypeptide from the HPV type 16 L2 protein is as shown in SEQ ID No. 4 or SEQ ID No. 5, the insertion site is the region of amino acids 426-429 of the N-terminus truncated mutants of the HPV type 58 L1 protein, after deleting the region of amino acids 427-428, the polypeptide from the HPV type 16 L2 protein is inserted between amino acids 426 and 429, and the amino acid sequence of the obtained papillomavirus chimeric protein is as shown in SEQ ID No. 11, SEQ ID No. 12, SEQ ID No. 13, SEQ ID No. 14, SEQ ID No. 15, SEQ ID No. 16 or SEQ ID No. 17.

Alternatively, in an embodiment of insertion by non-isometric substitution, the amino acid sequence of the polypeptide from the HPV type 16 L2 protein is as shown in SEQ ID No. 3, the insertion site is the region of amino acids 412-426 of the HPV type 58 L1 protein or the mutant of the HPV type 58 L1 protein, after deleting the region of amino acids 413-425, the polypeptide from the HPV type 16 L2 protein is inserted between amino acids 412 and 426, and the amino acid sequence of the obtained papillomavirus chimeric protein is as shown in SEQ ID No. 18 or SEQ ID No. 19.

Alternatively, the polypeptide from the HPV type 16 L2 protein is inserted into the surface region of the mutant of the HPV type 58 L1 protein by direct insertion or insertion by non-isometric substitution, the mutant of the HPV type 58 L1 protein is selected from the group consisting of:
a mutant with a truncation of amino acids at positions 2-4 at the N-terminus and/or a 25-amino acid truncation at the C-terminus of the amino acid sequence as shown in SEQ ID No. 1;
a mutant (CS1) with a truncation of amino acids at positions 2-4 at the N-terminus of the amino acid sequence as shown in SEQ ID No. 1 and substitution of amino acids 476, 481, 492, 493, 497 to glycine (G), amino acids 478, 487, 494, 498 to serine (S) and amino acids 480 and 495 to alanine (A) in the amino acid sequence as shown in SEQ ID No. 1;
a mutant (CS2) with a truncation of amino acids at positions 2-4 at the N-terminus of the amino acid sequence as shown in SEQ ID No. 1 and substitution of amino acids 474, 476, 481, 492, 493, 497 to glycine (G), amino acids 478, 487, 494, 498 to serine (S) and amino acids 480 and 495 to alanine (A) in the amino acid sequence as shown in SEQ ID No. 1; and
a mutant (CS3) with a truncation of amino acids at positions 2-4 at the N-terminus of the amino acid sequence as shown in SEQ ID No. 1 and substitution of amino acids 476, 481, 492, 493, 497 to glycine (G), amino acids 478, 494, 498 to serine (S) and amino acids 480 and 495 to alanine (A) in the amino acid sequence as shown in SEQ ID No. 1.

In another aspect, the present invention relates to a polynucleotide encoding the above human papillomavirus chimeric protein.

The present invention also provides a vector comprising the above polynucleotide, as well as a cell comprising the vector.

The polynucleotide sequence encoding the above human papillomavirus chimeric protein of the present invention is suitable for different expression systems. Alternatively, these nucleotide sequences are whole-gene optimized with E. coli codons and can be expressed at high levels in an E. coli expression system; or, they are whole-gene optimized with insect cell codons and can be expressed at high levels in an insect cell expression system.

The present invention also provides a polymer, preferably, the polymer is a pentamer or chimeric virus-like particle formed by the human papillomavirus chimeric protein of the present invention, wherein the polymer comprises the human papillomavirus chimeric protein according to the present invention, or is formed by the human papillomavirus chimeric protein according to the present invention.

The present invention also provides use of the human papillomavirus chimeric protein, the pentamer or the virus-like particle formed by the human papillomavirus chimeric protein in the preparation of a vaccine for the prevention of human papillomavirus infection and infection-induced diseases.

The present invention relates to human papillomavirus infection-induced diseases, including but not limited to cervical intraepithelial neoplasia, cervical cancer, vulval cancer, penile cancer, vaginal cancer, perianal cancer, oropharyngeal cancer, perianal and genital condylomata acuminata, respiratory recurrent papilloma, skin verrucous hyperplasia, skin squamous cell carcinoma and basal cell carcinoma. In some embodiments, the human papillomavirus infection is related to a virus selected from the group consisting of the oncogenic types HPV16, HPV18, HPV26, HPV31, HPV33, HPV35, HPV39, HPV45, HPV51, HPV52, HPV53, HPV56, HPV58, HPV59, HPV66, HPV68, HPV70, HPV73; the low-risk types HPV6, HPV11; as well as the skin types HPV2, HPVS, HPV27, HPV57.

The present invention also provides a vaccine for the prevention of human papillomavirus infection or infection-induced diseases, comprising:
1) the human papillomavirus chimeric protein of the present invention, and/or a pentamer or a virus-like particle formed by the human papillomavirus chimeric protein of the present invention;
2) optionally, an adjuvant;
3) optionally, an excipient or carrier for use in vaccines;
4) preferably, further comprising at least one virus-like particle or chimeric virus-like particle of mucosa-tropic and/or skin-tropic HPV.

In some embodiments, the content of the above virus-like particles in the above vaccine is an effective amount that can separately induce a protective immune response.

In some embodiments, the adjuvant is an adjuvant for human use. Preferably, the adjuvant includes, but is not limited to, aluminum adjuvant; an adjuvant composition of oil-in-water emulsion or water-in-oil emulsion and TLR stimulant; a composition of aluminum hydroxide adjuvant or aluminum phosphate adjuvant with polyinosinic acid-polycytidylic acid adjuvant and a stabilizer; or a composition of MF59 adjuvant with polyinosinic acid-polycytidylic acid adjuvant and a stabilizer.

In some embodiments, the vaccine of the present invention can be in a patient-acceptable form, including but not limited to oral administration or injection, preferably injection.

In some embodiments, the vaccine of the present invention is preferably prepared in a unit dosage form, wherein the dose of the human papillomavirus chimeric protein or protein virus-like particles in the unit dosage form is 5 μg to 100 for example, 5, 10, 15, 20, 25, 30, 35, 40, 45, 50, 55, 60, 65, 70, 75, 80, 85, 90, 95, 100 as well as the range between any two of the above values, preferably 30 μg to 60 μg per unit dosage form.

Description and Explanation of Relevant Terms in the Invention

According to the present invention, the term “insect cell expression system” includes insect cell, recombinant baculovirus, recombinant Bacmid and expression vector. Among them, the insect cell is derived from commercially available cells, the examples of which are listed here but are not limited to: Sf9, Sf21, High Five.

According to the present invention, the term “prokaryotic expression system” includes but is not limited to E. coli expression system. Wherein, the expression host bacteria are derived from commercially available strains, the examples of which are listed here but are not limited to: BL21 (DE3), BL21 (DE3) plysS, C43 (DE3), Rosetta-gami B (DE3).

According to the present invention, examples of the term “wild-type HPV type 58 L1 protein” include, but are not limited to the protein No. CAX48979.1 in the NCBI database.

The gene fragment of “mutant of the HPV type 58 L1 protein” means that it has a deletion of nucleotides encoding one or more amino acids at its 5′ end and/or 3′ end compared with the gene encoding the wild-type HPV type 58 L1 protein, and/or nucleotide mutations leading to amino acid mutations exist at one or more sites in its sequence, wherein the full-length sequence of “wild-type HPV type 58 L1 protein” is for example, but is not limited to, the following sequences in the NCBI database: AFS33402.1, ADK78323.1, AMY16498.1, ACJ13512.1, ADK78590.1, ADK78685.1, etc.

According to the present invention, the term “excipient or carrier for use in vaccines” refers to one or more selected from the following, including but not limited to: pH adjuster, surfactant and ionic strength enhancer. For example, the pH adjuster is for example but not limited to phosphate buffered saline. The surfactant includes cationic, anionic or nonionic surfactant, and is for example but not limited to polysorbate 80 (Tween-80). The ionic strength enhancer is for example but not limited to sodium chloride.

According to the present invention, the term “adjuvant for use in human” refers to an adjuvant that can be applied clinically to the human body, including various adjuvants that have been approved and may be approved in the future, for example, but not limited to, aluminum adjuvant, MF59 and various forms of adjuvant compositions.

According to the present invention, the term “emulsion” refers to a heterogeneous liquid dispersion system formed after emulsification by mixing an aqueous phase component, an oil phase component and an emulsifier at an appropriate ratio. Wherein, the aqueous phase component includes but is not limited to phosphate buffered saline, HEPES buffer and other buffer systems; the oil-phase component is a metabolizable lipid, including but not limited to vegetable oil, fish oil, animal oil, synthetic oil and other lipid component (for example but not limited to squalene, tocopherol). The emulsifier is a suitable surfactant, for example but not limited to sorbitan trioleate (Span-85), polysorbate 80 (Tween-80).

According to the present invention, the term “stabilizer” refers to a component that can bind to polyinosinic acid-polycytidylic acid in the adjuvant and play a stabilizing role, including but not limited to antibiotics (for example but not limited to kanamycin, neomycin, gentamicin), inorganic salts (for example but not limited to calcium chloride, magnesium chloride, calcium phosphate), cationic organic complexes (for example but not limited to calcium stearate, calcium gluconate).

### DESCRIPTION OF THE DRAWINGS

FIG. 1A to FIG. 1B: Identification of the expression of the chimeric proteins in Example 5 of the present invention in E. coli and insect cells. The results showed that the chimeric proteins could all be expressed at high levels in E. coli and insect cells.

FIG. 1A: Identification of the expression of chimeric proteins in E. coli, 1 to 5 representing 58L1DE/16dEs, 58L1h4/16dEs, 58L1ΔN4h4/16dE, 58L1ΔN4h4/16dEs, and 58L1h4/16dE, respectively.

FIG. 1B: Identification of the expression of chimeric proteins in insect cells, 1 to 8 representing 58L1ΔCDE/16dEs, 58L1ΔCh4/16dEs, 58L1ΔN4Ch4/16dE, 58L1ΔN4Ch4/16dEs, 58L1ΔCh4/16dE, 58L1ΔN4h4/16dE-CS1, 58L1ΔN4h4/16dE-CS2, and 58L1ΔN4h4/16dE-CS3, respectively.

FIG. 2A to FIG. 2E: Dynamic light scattering analysis results of chimeric particles or cVLPs obtained after purification in Example 6 of the present invention. The results showed that the hydraulic diameters of the virus-like particles formed by 58L1ΔCDE/16dEs, 58L1ΔCh4/16dEs, 58L1ΔN4Ch4/16dE and 58L1ΔN4Ch4/16dEs recombinant proteins were 90 nm, 114.6 nm, 83.5 nm and 93 nm, respectively, and the percentages of particle assembly were all 100%. The hydraulic diameter of the particle formed by 58L1ΔCh4/16dE recombinant protein was 12.28 nm.

FIG. 2A: Dynamic light scattering analysis results of cVLPs obtained from 58L1ΔCDE/16dEs;

FIG. 2B: Dynamic light scattering analysis results of cVLPs obtained from 58L1ΔCh4/16dEs;

FIG. 2C: Dynamic light scattering analysis results of cVLPs obtained from 58L1ΔN4Ch4/16dE;

FIG. 2D: Dynamic light scattering analysis results of cVLPs obtained from 58L1ΔN4Ch4/16dEs;

FIG. 2E: Dynamic light scattering analysis results of cVLPs obtained from 58L1ΔCh4/16dE.

FIG. 3A to FIG. 3D: Transmission electron microscopy observation results of cVLPs obtained after purification in Example 7 of the present invention. A large number of virus-like particles could be seen in the field, and the particles had good uniformity. The diameter of cVLPs was about 50 nm. Bar=100 nm.

FIG. 3A: Transmission electron microscopy observation results of cVLPs obtained from 58L1ΔCDE/16dEs;

FIG. 3B: Transmission electron microscopy observation results of cVLPs obtained from 58L1ΔCh4/16dEs;

FIG. 3C: Transmission electron microscopy observation results of cVLPs obtained from 58L1ΔN4Ch4/16dE;

FIG. 3D: Transmission electron microscopy observation results of cVLPs obtained from 58L1ΔN4Ch4/16dEs.

### DETAILED DESCRIPTION OF THE INVENTION

The present invention will be further illustrated by the non-limiting examples below. It is well known to those skilled in the art that many modifications can be made to the present invention without departing from the spirit of the present invention, and such modifications also fall within the scope of the present invention. The following embodiments are only used to illustrate the present invention and should not be regarded as limiting the scope of the present invention, as the embodiments are necessarily diverse. The terms used in the present specification are intended only to describe particular embodiments but not as limitations. The scope of the present invention has been defined in the appended claims.

Unless otherwise specified, all the technical and scientific terms used in the present specification have the same meaning as those generally understood by those skilled in the technical field to which the present application relates. Preferred methods and materials of the present invention are described below, but any method and material similar or equivalent to the methods and materials described in the present specification can be used to implement or test the present invention. Unless otherwise specified, the following experimental methods are conventional methods or methods described in product specifications. Unless otherwise specified, the experimental materials used are easily available from commercial companies. All published literatures referred to in the present specification are incorporated here by reference to reveal and illustrate the methods and/or materials in the published literatures.

### Example 1: Synthesis of Chimeric Protein Genes and Construction of Expression Vectors

There were 13 types of chimeric proteins, namely:
1) Chimeric protein 58L1DE/16dEs: the backbone was full-length HPV type 58 L1 protein (the sequence was as shown in SEQ ID No. 1), where the polypeptide of aa. 19-31 of HPV type 16 L2 protein (the amino acid sequence was as shown in SEQ ID No. 6) was fused between aa. 136/137. The amino acid sequence of the chimeric protein 58L1DE/16dEs was as shown in SEQ ID No. 7. The polynucleotide sequence encoding 58L1DE/16dEs was optimized with E. coli codons, and its sequence was as shown in SEQ ID No. 20;
2) Chimeric protein 58L1h4/16dEs: the backbone was full-length HPV type 58 L1 protein (the sequence was as shown in SEQ ID No. 1), where the region of aa. 430-431 was deleted, and the polypeptide of aa. 19-31 of HPV type 16 L2 protein (the amino acid sequence was as shown in SEQ ID No. 6) was fused between aa. 429/432. The amino acid sequence of the chimeric protein 58L1h4/16dEs was as shown in SEQ ID No. 9. The polynucleotide sequence encoding 58L1h4/16dEs was optimized with E. coli codons, and its sequence was as shown in SEQ ID No. 22;
3) Chimeric protein 58L1ΔN4h4/16dE: the backbone was HPV type 58 L1 protein with a deletion of amino acids at positions 2-4 at the N-terminus (the sequence was as shown in SEQ ID No. 1 with a deletion of amino acids at positions 2-4 at the N-terminus), where the region of aa. 427-428 was deleted, and the polypeptide of aa. 18-38 of HPV type 16 L2 protein (the amino acid sequence was as shown in SEQ ID No. 4) was fused between aa. 426/429. The amino acid sequence of the chimeric protein 58L1ΔN4h4/16dE was as shown in SEQ ID No. 11. The polynucleotide sequence encoding 58L1ΔN4h4/16dE was optimized with E. coli codons, and its sequence was as shown in SEQ ID No. 24;
4) Chimeric protein 58L1ΔN4h4/16dEs: the backbone was HPV type 58 L1 protein with a deletion of amino acids at positions 2-4 at the N-terminus (the sequence was as shown in SEQ ID No. 1 with a deletion of amino acids at positions 2-4 at the N-terminus), where the region of aa. 427-428 was deleted, and the polypeptide of aa. 18-32 of HPV type 16 L2 protein (the amino acid sequence was as shown in SEQ ID No. 5) was fused between aa. 426/429. The amino acid sequence of the chimeric protein 58L1ΔN4h4/16dEs was as shown in SEQ ID No. 13. The polynucleotide sequence encoding 58L1ΔN4h4/16dEs was optimized with E. coli codons, and its sequence was as shown in SEQ ID No. 26;
5) Chimeric protein 58L1h4/16dE: the backbone was full-length HPV type 58 L1 protein (the sequence was as shown in SEQ ID No. 1), where the region of aa. 413-425 was deleted, and the polypeptide of aa. 17-38 of HPV type 16 L2 protein (the amino acid sequence was as shown in SEQ ID No. 3) was fused between aa. 412/426. The amino acid sequence of the chimeric protein 58L1h4/16dE was as shown in SEQ ID No. 18. The polynucleotide sequence encoding 58L1h4/16dEs was optimized with E. coli codons, and its sequence was as shown in SEQ ID No. 31;
6) Chimeric protein 58L1ΔCDE/16dEs: the backbone was HPV type 58 L1 protein with a 25-amino acid truncation at the C-terminus (SEQ ID No. 1 with a 25-amino acid truncation at the C-terminus), where the polypeptide of aa. 19-31 of HPV type 16 L2 protein (the amino acid sequence was as shown in SEQ ID No. 6) was fused between aa. 136/137. The amino acid sequence of the chimeric protein 58L1ΔCDE/16dEs was as shown in SEQ ID No. 8. The polynucleotide sequence encoding 58L1ΔCDE/16dEs was optimized with Sf9 insect cell codons, and its sequence was as shown in SEQ ID No. 21;
7) Chimeric protein 58L1ΔCh4/16dEs: the backbone was HPV type 58 L1 protein with a 25-amino acid truncation at the C-terminus (SEQ ID No. 1 with a 25-amino acid truncation at the C-terminus), where the region of aa. 430-431 was deleted, and the polypeptide of aa. 19-31 of HPV type 16 L2 protein (the amino acid sequence was as shown in SEQ ID No. 6) was fused between aa. 429/432. The amino acid sequence of the chimeric protein 58L1ΔCh4/16dEs was as shown in SEQ ID No. 10. The polynucleotide sequence encoding 58L1ΔCh4/16dEs was optimized with 519 insect cell codons, and its sequence was as shown in SEQ ID No. 23;
8) Chimeric protein 58L1ΔN4Ch4/16dE: the backbone was HPV type 58 L1 protein with a deletion of amino acids at positions 2-4 at the N-terminus and a 25-amino acid truncation at the C-terminus (SEQ ID No. 1 with a deletion of amino acids at positions 2-4 at the N-terminus and a 25-amino acid truncation at the C-terminus), where the region of aa. 427-428 was deleted, and the polypeptide of aa. 18-38 of HPV type 16 L2 protein (the amino acid sequence was as shown in SEQ ID No. 4) was fused between aa. 426/429. The amino acid sequence of 58L1ΔN4Ch4/16dE was as shown in SEQ ID No. 12. The polynucleotide sequence encoding 58L1ΔN4Ch4/16dE was optimized with Sf9 insect cell codons, and its sequence was as shown in SEQ ID No. 25;
9) Chimeric protein 58L1ΔN4Ch4/16dEs: the backbone was HPV type 58 L1 protein with a deletion of amino acids at positions 2-4 at the N-terminus and a 25-amino acid truncation at the C-terminus (SEQ ID No. 1 with a deletion of amino acids at positions 2-4 at the N-terminus and a 25-amino acid truncation at the C-terminus), where the region of aa. 427-428 was deleted, and the polypeptide of aa. 18-32 of HPV type 16 L2 protein (the amino acid sequence was as shown in SEQ ID No. 5) was fused between aa. 426/429. The amino acid sequence of the chimeric protein 58L1ΔN4Ch4/16dEs was as shown in SEQ ID No. 14. The polynucleotide sequence encoding 58L1ΔN4Ch4/16dEs was optimized with Sf9 insect cell codons, and its sequence was as shown in SEQ ID No. 27;
10) Chimeric protein 58L1ΔN4h4/16dE-CS1: the backbone was the mutant with a deletion of amino acids at positions 2-4 at the N-terminus of the amino acid sequence as shown in SEQ ID No. 1 and substitutions of amino acids 476, 481, 492, 493, 497 to glycine (G), amino acids 478, 487, 494, 498 to serine (S) and amino acids 480 and 495 to alanine (A) in the amino acid sequence as shown in SEQ ID No. 1, where the region of aa. 427-428 was deleted, and the polypeptide of aa. 18-38 of HPV type 16 L2 protein (the amino acid sequence was as shown in SEQ ID No. 4) was fused between aa. 426/429. The amino acid sequence of 58L1ΔN4Ch4/16dE-CS1 was as shown in SEQ ID No. 15. The polynucleotide sequence encoding 58L1ΔN4Ch4/16dE-CS1 was optimized with Sf9 insect cell codons, and its sequence was as shown in SEQ ID No. 28;
11) Chimeric protein 58L1ΔN4h4/16dE-052: the backbone was the mutant with a deletion of amino acids at positions 2-4 at the N-terminus of the amino acid sequence as shown in SEQ ID No. 1 and substitutions of amino acids 474, 476, 481, 492, 493, 497 to glycine (G), amino acids 478, 487, 494, 498 to serine (S) and amino acids 480 and 495 to alanine (A) in the amino acid sequence as shown in SEQ ID No. 1, where the region of aa. 427-428 was deleted, and the polypeptide of aa. 18-38 of HPV type 16 L2 protein (the amino acid sequence was as shown in SEQ ID No. 4) was fused between aa. 426/429. The amino acid sequence of 58L1ΔN4Ch4/16dE-052 was as shown in SEQ ID No. 16. The polynucleotide sequence encoding 58L1ΔN4Ch4/16dE-052 was optimized with Sf9 insect cell codons, and its sequence was as shown in SEQ ID No. 29;
12) Chimeric protein 58L1ΔN4h4/16dE-053: the backbone was the mutant with a deletion of amino acids at positions 2-4 at the N-terminus of the amino acid sequence as shown in SEQ ID No. 1 and substitutions of amino acids 476, 481, 492, 493, 497 to glycine (G), amino acids 478, 494, 498 to serine (S) and amino acids 480 and 495 to alanine (A) in the amino acid sequence as shown in SEQ ID No. 1, where the region of aa. 427-428 was deleted, and the polypeptide of aa. 18-38 of HPV type 16 L2 protein (the amino acid sequence was as shown in SEQ ID No. 4) was fused between aa. 426/429. The amino acid sequence of 58L1ΔN4Ch4/16dE-053 was as shown in SEQ ID No. 17. The polynucleotide sequence encoding 58L1ΔN4Ch4/16dE-053 was optimized with Sf9 insect cell codons, and its sequence was as shown in SEQ ID No. 30;
13) Chimeric protein 58L1ΔCh4/16dE: the backbone was HPV type 58 L1 protein with a 25-amino acid truncation at the C-terminus (SEQ ID No. 1 with a 25-amino acid truncation at the C-terminus), where the region of aa. 413-425 was deleted, and the polypeptide of aa. 17-38 of HPV type 16 L2 protein (the amino acid sequence was as shown in SEQ ID No. 3) was fused between aa. 412/426. The amino acid sequence of 58L1ΔCh4/16dE was as shown in SEQ ID No. 19. The polynucleotide sequence encoding 58L1ΔCh4/16dE was optimized with Sf9 insect cell codons, and its sequence was as shown in SEQ ID No. 32.

The chimeric L1 genes optimized according to E. coli codons and according to insect cell codons were synthesized by Shanghai Sangon Biotech Co., Ltd. by whole-gene synthesis.

The chimeric protein genes optimized with E. coli codons were digested by NdeI/XhoI and inserted into the commercial expression vector pET22b (produced by Novagen), respectively.

The chimeric protein genes optimized with insect cell codons were digested by EcoRI/XbaI and inserted into the commercial expression vector pFastBac1 (produced by Invitrogen), respectively.

Expression vectors comprising the chimeric protein genes were obtained, namely:
pET22b-58L1DE/16dEs;
pET22b-58L1h4/16dEs;
pET22b-58L1ΔN4h4/16dE;
pET22b-58L1ΔN4h4/16dEs;
pET22b-58L1h4/16dE;
pFastBac1-58L1ΔCDE/16dEs;
pFastBac1-58L1ΔCh4/16dEs;
pFastBac1-58L1ΔN4Ch4/16dE;
pFastBac1-58L1ΔN4Ch4/16dEs;
pFastBac1-58L1ΔN4h4/16dE-CS1;
pFastBac1-58L1ΔN4h4/16dE-CS2;
pFastBac1-58L1ΔN4h4/16dE-CS3;
pFastBac1-58L1ΔCh4/16dE.

The above methods of enzyme digestion, ligation and construction of clones were all well known, for example, the patent CN 101293918 B.

The amino acid sequences of the L1, L2 proteins and chimeric proteins involved in the present invention were as shown below:

Full length of type 58 L1 protein
SEQ ID No. 1
MSVWRPSEAT VYLPPVPVSK VVSTDEYVSR TSIYYYAGSS RLLAVGNPYF
SIKSPNNNKK VLVPKVSGLQ YRVFRVRLPD PNKFGFPDTS FYNPDTQRLV
WACVGLEIGR GQPLGVGVSG HPYLNKFDDT ETSNRYPAQP GSDNRECLSM
DYKQTQLCLI GCKPPTGEHW GKGVACNNNA AATDCPPLEL FNSIIEDGDM
VDTGFGCMDF GTLQANKSDV PIDICNSTCK YPDYLKMASE PYGDSLFFFL
RREQMFVRHF FNRAGKLGEA VPDDLYIKGS GNTAVIQSSA FFPTPSGSIV
TSESQLFNKP YWLQRAQGHN NGICWGNQLF VTVVDTTRST NMTLCTEVTK
EGTYKNDNFK EYVRHVEEYD LQFVFQLCKI TLTAEIMTYI HTMDSNILED
WQFGLTPPPS ASLQDTYRFV TSQAITCQKT APPKEKEDPL NKYTFWEVNL
KEKFSADLDQ FPLGRKFLLQ SGLKAKPRLK RSAPTTRAPS TKRKKVKK
Full length of type 16 L2 protein
SEQ ID No. 2
MRHKRSAKRTKRASATQLYKTCKQAGTCPPDIIPKVEGKTIAEQILQYGSMGVFFGGLGIGTGSGTGGRTGYIPLGT
RPPTATDTLAPVRPPLTVDPVGPSDPSIVSLVEETSFIDAGAPTSVPSIPPDVSGFSITTSTD
TTPAILDINNTVTTVTTHNNPTFTDPSVLQPPTPAETGGHFTLSSSTISTHNYEEIPMDTFIVSTNPNTV
TSSTPIPGSRPVARLGLYSRTTQQVKVVDPAFVTTPTKLITYDNPAYEGIDVDNTLYFSSNDNSINIAPD
PDFLDIVALHRPALTSRRTGIRYSRIGNKQTLRTRSGKSIGAKVHYYYDLSTIDPAEEIELQTITPSTYT
TTSHAASPTSINNGLYDIYADDFITDTSTTPVPSVPSTSLSGYIPANTTIPFGGAYNIPLVSGPDIPINI
TDQAPSLIPIVPGSPQYTIIADAGDFYLHPSYYMLRKRRKRLPYFFSDVSLAA
16L2 aa. 17-38
SEQ ID No. 3
QLYKTCKQAGTCPPDIIPKVEG
16L2 aa. 18-38
SEQ ID No. 4
LYKTCKQAGT CPPDIIPKVE G
16L2 aa. 18-32
SEQ ID No. 5
LYKTCKQAGT CPPDI
16L2 aa.19-31
SEQ ID No. 6
YKTCKQAGTC PPD
58L1DE/16dEs
SEQ ID No. 7
MSVWRPSEAT VYLPPVPVSK VVSTDEYVSR TSIYYYAGSS RLLAVGNPYF SIKSPNNNKK
VLVPKVSGLQ YRVFRVRLPD PNKFGFPDTS FYNPDTQRLV WACVGLEIGR GQPLGVGVSG
HPYLNKFDDT ETSNRYYKTC KQAGTCPPDP AQPGSDNREC LSMDYKQTQL CLIGCKPPTG
EHWGKGVACN NNAAATDCPP LELFNSIIED GDMVDTGFGC MDFGTLQANK SDVPIDICNS
TCKYPDYLKM ASEPYGDSLF FFLRREQMFV RHFFNRAGKL GEAVPDDLYI KGSGNTAVIQ
SSAFFPTPSG SIVTSESQLF NKPYWLQRAQ GHNNGICWGN QLFVTVVDTT RSTNMTLCTE
VTKEGTYKND NFKEYVRHVE EYDLQFVFQL CKITLTAEIM TYIHTMDSNI LEDWQFGLTP
PPSASLQDTY RFVTSQAITC QKTAPPKEKE DPLNKYTFWE VNLKEKFSAD LDQFPLGRKF
LLQSGLKAKP RLKRSAPTTR APSTKRKKVK K
58L1ΔCDE/16dEs
SEQ ID No. 8
MSVWRPSEAT VYLPPVPVSK VVSTDEYVSR TSIYYYAGSS RLLAVGNPYF SIKSPNNNKK
VLVPKVSGLQ YRVFRVRLPD PNKFGFPDTS FYNPDTQRLV WACVGLEIGR GQPLGVGVSG
HPYLNKFDDT ETSNRYYKTC KQAGTCPPDP AQPGSDNREC LSMDYKQTQL CLIGCKPPTG
EHWGKGVACN NNAAATDCPP LELFNSIIED GDMVDTGFGC MDFGTLQANK SDVPIDICNS
TCKYPDYLKM ASEPYGDSLF FFLRREQMFV RHFFNRAGKL GEAVPDDLYI KGSGNTAVIQ
SSAFFPTPSG SIVTSESQLF NKPYWLQRAQ GHNNGICWGN QLFVTVVDTT RSTNMTLCTE
VTKEGTYKND NFKEYVRHVE EYDLQFVFQL CKITLTAEIM TYIHTMDSNI LEDWQFGLTP
PPSASLQDTY RFVTSQAITC QKTAPPKEKE DPLNKYTFWE VNLKEKFSAD LDQFPLGRKF
LLQSGL
58L1h4/16dEs
SEQ ID No. 9
MSVWRPSEAT VYLPPVPVSK VVSTDEYVSR TSIYYYAGSS RLLAVGNPYF SIKSPNNNKK
VLVPKVSGLQ YRVFRVRLPD PNKFGFPDTS FYNPDTQRLV WACVGLEIGR GQPLGVGVSG
HPYLNKFDDT ETSNRYPAQP GSDNRECLSM DYKQTQLCLI GCKPPTGEHW GKGVACNNNA
AATDCPPLEL FNSIIEDGDM VDTGFGCMDF GTLQANKSDV PIDICNSTCK YPDYLKMASE
PYGDSLFFFL RREQMFVRHF FNRAGKLGEA VPDDLYIKGS GNTAVIQSSA FFPTPSGSIV
TSESQLFNKP YWLQRAQGHN NGICWGNQLF VTVVDTTRST NMTLCTEVTK EGTYKNDNFK
EYVRHVEEYD LQFVFQLCKI TLTAEIMTYI HTMDSNILED WQFGLTPPPS ASLQDTYRFV
TSQAITCQKY KTCKQAGTCP PDPPKEKEDP LNKYTFWEVN LKEKFSADLD QFPLGRKFLL
QSGLKAKPRL KRSAPTTRAP STKRKKVKK
58L1ΔCh4/16dEs
SEQ ID No. 10
MSVWRPSEAT VYLPPVPVSK VVSTDEYVSR TSIYYYAGSS RLLAVGNPYF SIKSPNNNKK
VLVPKVSGLQ YRVFRVRLPD PNKFGFPDTS FYNPDTQRLV WACVGLEIGR GQPLGVGVSG
HPYLNKFDDT ETSNRYPAQP GSDNRECLSM DYKQTQLCLI GCKPPTGEHW GKGVACNNNA
AATDCPPLEL FNSIIEDGDM VDTGFGCMDF GTLQANKSDV PIDICNSTCK YPDYLKMASE
PYGDSLFFFL RREQMFVRHF FNRAGKLGEA VPDDLYIKGS GNTAVIQSSA FFPTPSGSIV
TSESQLFNKP YWLQRAQGHN NGICWGNQLF VTVVDTTRST NMTLCTEVTK EGTYKNDNFK
EYVRHVEEYD LQFVFQLCKI TLTAEIMTYI HTMDSNILED WQFGLTPPPS ASLQDTYRFV
TSQAITCQKY KTCKQAGTCP PDPPKEKEDP LNKYTFWEVN LKEKFSADLD QFPLGRKFLL
QSGL
58L1ΔN4h4/16dE
SEQ ID No. 11
MRPSEATVYL PPVPVSKVVS TDEYVSRTSI YYYAGSSRLL AVGNPYFSIK SPNNNKKVLV
PKVSGLQYRV FRVRLPDPNK FGFPDTSFYN PDTQRLVWAC VGLEIGRGQP LGVGVSGHPY
LNKFDDTETS NRYPAQPGSD NRECLSMDYK QTQLCLIGCK PPTGEHWGKG VACNNNAAAT
DCPPLELFNS IIEDGDMVDT GFGCMDFGTL QANKSDVPID ICNSTCKYPD YLKMASEPYG
DSLFFFLRRE QMFVRHFFNR AGKLGEAVPD DLYIKGSGNT AVIQSSAFFP TPSGSIVTSE
SQLFNKPYWL QRAQGHNNGI CWGNQLFVTV VDTTRSTNMT LCTEVTKEGT YKNDNFKEYV
RHVEEYDLQF VFQLCKITLT AEIMTYIHTM DSNILEDWQF GLTPPPSASL QDTYRFVTSQ
AITCQKLYKT CKQAGTCPPD IIPKVEGPPK EKEDPLNKYT FWEVNLKEKF SADLDQFPLG
RKFLLQSGLK AKPRLKRSAP TTRAPSTKRK KVKK
58L1ΔN4Ch4/16dE
SEQ ID No. 12
MRPSEATVYL PPVPVSKVVS TDEYVSRTSI YYYAGSSRLL AVGNPYFSIK SPNNNKKVLV
PKVSGLQYRV FRVRLPDPNK FGFPDTSFYN PDTQRLVWAC VGLEIGRGQP LGVGVSGHPY
LNKFDDTETS NRYPAQPGSD NRECLSMDYK QTQLCLIGCK PPTGEHWGKG VACNNNAAAT
DCPPLELFNS IIEDGDMVDT GFGCMDFGTL QANKSDVPID ICNSTCKYPD YLKMASEPYG
DSLFFFLRRE QMFVRHFFNR AGKLGEAVPD DLYIKGSGNT AVIQSSAFFP TPSGSIVTSE
SQLFNKPYWL QRAQGHNNGI CWGNQLFVTV VDTTRSTNMT LCTEVTKEGT YKNDNFKEYV
RHVEEYDLQF VFQLCKITLT AEIMTYIHTM DSNILEDWQF GLTPPPSASL QDTYRFVTSQ
AITCQKLYKT CKQAGTCPPD IIPKVEGPPK EKEDPLNKYT FWEVNLKEKF SADLDQFPLG
RKFLLQSGL
58L1ΔN4h4/16dEs
SEQ ID No. 13
MRPSEATVYL PPVPVSKVVS TDEYVSRTSI YYYAGSSRLL AVGNPYFSIK SPNNNKKVLV
PKVSGLQYRV FRVRLPDPNK FGFPDTSFYN PDTQRLVWAC VGLEIGRGQP LGVGVSGHPY
LNKFDDTETS NRYPAQPGSD NRECLSMDYK QTQLCLIGCK PPTGEHWGKG VACNNNAAAT
DCPPLELFNS IIEDGDMVDT GFGCMDFGTL QANKSDVPID ICNSTCKYPD YLKMASEPYG
DSLFFFLRRE QMFVRHFFNR AGKLGEAVPD DLYIKGSGNT AVIQSSAFFP TPSGSIVTSE
SQLFNKPYWL QRAQGHNNGI CWGNQLFVTV VDTTRSTNMT LCTEVTKEGT YKNDNFKEYV
RHVEEYDLQF VFQLCKITLT AEIMTYIHTM DSNILEDWQF GLTPPPSASL QDTYRFVTSQ
AITCQKLYKT CKQAGTCPPD IPPKEKEDPL NKYTFWEVNL KEKFSADLDQ FPLGRKFLLQ
SGLKAKPRLK RSAPTTRAPS TKRKKVKK
58L1ΔN4Ch4/16dEs
SEQ ID No. 14
MRPSEATVYL PPVPVSKVVS TDEYVSRTSI YYYAGSSRLL AVGNPYFSIK SPNNNKKVLV
PKVSGLQYRV FRVRLPDPNK FGFPDTSFYN PDTQRLVWAC VGLEIGRGQP LGVGVSGHPY
LNKFDDTETS NRYPAQPGSD NRECLSMDYK QTQLCLIGCK PPTGEHWGKG VACNNNAAAT
DCPPLELFNS IIEDGDMVDT GFGCMDFGTL QANKSDVPID ICNSTCKYPD YLKMASEPYG
DSLFFFLRRE QMFVRHFFNR AGKLGEAVPD DLYIKGSGNT AVIQSSAFFP TPSGSIVTSE
SQLFNKPYWL QRAQGHNNGI CWGNQLFVTV VDTTRSTNMT LCTEVTKEGT YKNDNFKEYV
RHVEEYDLQF VFQLCKITLT AEIMTYIHTM DSNILEDWQF GLTPPPSASL QDTYRFVTSQ
AITCQKLYKT CKQAGTCPPD IPPKEKEDPL NKYTFWEVNL KEKFSADLDQ FPLGRKFLLQ
SGL
58L1ΔN4h4/16dE-CS1
SEQ ID No. 15
MRPSEATVYL PPVPVSKVVS TDEYVSRTSI YYYAGSSRLL AVGNPYFSIK SPNNNKKVLV
PKVSGLQYRV FRVRLPDPNK FGFPDTSFYN PDTQRLVWAC VGLEIGRGQP LGVGVSGHPY
LNKFDDTETS NRYPAQPGSD NRECLSMDYK QTQLCLIGCK PPTGEHWGKG VACNNNAAAT
DCPPLELFNS IIEDGDMVDT GFGCMDFGTL QANKSDVPID ICNSTCKYPD YLKMASEPYG
DSLFFFLRRE QMFVRHFFNR AGKLGEAVPD DLYIKGSGNT AVIQSSAFFP TPSGSIVTSE
SQLFNKPYWL QRAQGHNNGI CWGNQLFVTV VDTTRSTNMT LCTEVTKEGT YKNDNFKEYV
RHVEEYDLQF VFQLCKITLT AEIMTYIHTM DSNILEDWQF GLTPPPSASL QDTYRFVTSQ
AITCQKLYKT CKQAGTCPPD IIPKVEGPPK EKEDPLNKYT FWEVNLKEKF SADLDQFPLG
RKFLLQSGLK AGPSLAGSAP TTSAPSTGGS AVGS
58L1ΔN4h4/16dE-CS2
SEQ ID No. 16
MRPSEATVYL PPVPVSKVVS TDEYVSRTSI YYYAGSSRLL AVGNPYFSIK SPNNNKKVLV
PKVSGLQYRV FRVRLPDPNK FGFPDTSFYN PDTQRLVWAC VGLEIGRGQP LGVGVSGHPY
LNKFDDTETS NRYPAQPGSD NRECLSMDYK QTQLCLIGCK PPTGEHWGKG VACNNNAAAT
DCPPLELFNS IIEDGDMVDT GFGCMDFGTL QANKSDVPID ICNSTCKYPD YLKMASEPYG
DSLFFFLRRE QMFVRHFFNR AGKLGEAVPD DLYIKGSGNT AVIQSSAFFP TPSGSIVTSE
SQLFNKPYWL QRAQGHNNGI CWGNQLFVTV VDTTRSTNMT LCTEVTKEGT YKNDNFKEYV
RHVEEYDLQF VFQLCKITLT AEIMTYIHTM DSNILEDWQF GLTPPPSASL QDTYRFVTSQ
AITCQKLYKT CKQAGTCPPD IIPKVEGPPK EKEDPLNKYT FWEVNLKEKF SADLDQFPLG
RKFLLQSGLG AGPSLAGSAP TTSAPSTGGS AVGS
58L1ΔN4h4/16dE-CS3
SEQ ID No. 17
MRPSEATVYL PPVPVSKVVS TDEYVSRTSI YYYAGSSRLL AVGNPYFSIK SPNNNKKVLV
PKVSGLQYRV FRVRLPDPNK FGFPDTSFYN PDTQRLVWAC VGLEIGRGQP LGVGVSGHPY
LNKFDDTETS NRYPAQPGSD NRECLSMDYK QTQLCLIGCK PPTGEHWGKG VACNNNAAAT
DCPPLELFNS IIEDGDMVDT GFGCMDFGTL QANKSDVPID ICNSTCKYPD YLKMASEPYG
DSLFFFLRRE QMFVRHFFNR AGKLGEAVPD DLYIKGSGNT AVIQSSAFFP TPSGSIVTSE
SQLFNKPYWL QRAQGHNNGI CWGNQLFVTV VDTTRSTNMT LCTEVTKEGT YKNDNFKEYV
RHVEEYDLQF VFQLCKITLT AEIMTYIHTM DSNILEDWQF GLTPPPSASL QDTYRFVTSQ
AITCQKLYKT CKQAGTCPPD IIPKVEGPPK EKEDPLNKYT FWEVNLKEKF SADLDQFPLG
RKFLLQSGLK AGPSLAGSAP TTRAPSTGGS AVGS
58L1h4/16dE
SEQ ID No. 18
MSVWRPSEAT VYLPPVPVSK VVSTDEYVSR TSIYYYAGSS RLLAVGNPYF SIKSPNNNKK
VLVPKVSGLQ YRVFRVRLPD PNKFGFPDTS FYNPDTQRLV WACVGLEIGR GQPLGVGVSG
HPYLNKFDDT ETSNRYPAQP GSDNRECLSM DYKQTQLCLI GCKPPTGEHW GKGVACNNNA
AATDCPPLEL FNSIIEDGDM VDTGFGCMDF GTLQANKSDV PIDICNSTCK YPDYLKMASE
PYGDSLFFFL RREQMFVRHF FNRAGKLGEA VPDDLYIKGS GNTAVIQSSA FFPTPSGSIV
TSESQLFNKP YWLQRAQGHN NGICWGNQLF VTVVDTTRST NMTLCTEVTK EGTYKNDNFK
EYVRHVEEYD LQFVFQLCKI TLTAEIMTYI HTMDSNILED WQFGLTPPPS ASQLYKTCKQ
AGTCPPDIIP KVEGTCQKTA PPKEKEDPLN KYTFWEVNLK EKFSADLDQF PLGRKFLLQS
GLKAKPRLKR SAPTTRAPST KRKKVKK
58L1ΔCh4/16dE
SEQ ID No. 19
MSVWRPSEAT VYLPPVPVSK VVSTDEYVSR TSIYYYAGSS RLLAVGNPYF SIKSPNNNKK
VLVPKVSGLQ YRVFRVRLPD PNKFGFPDTS FYNPDTQRLV WACVGLEIGR GQPLGVGVSG
HPYLNKFDDT ETSNRYPAQP GSDNRECLSM DYKQTQLCLI GCKPPTGEHW GKGVACNNNA
AATDCPPLEL FNSIIEDGDM VDTGFGCMDF GTLQANKSDV PIDICNSTCK YPDYLKMASE
PYGDSLFFFL RREQMFVRHF FNRAGKLGEA VPDDLYIKGS GNTAVIQSSA FFPTPSGSIV
TSESQLFNKP YWLQRAQGHN NGICWGNQLF VTVVDTTRST NMTLCTEVTK EGTYKNDNFK
EYVRHVEEYD LQFVFQLCKI TLTAEIMTYI HTMDSNILED WQFGLTPPPS ASQLYKTCKQ
AGTCPPDIIP KVEGTCQKTA PPKEKEDPLN KYTFWEVNLK EKFSADLDQF PLGRKFLLQS
GL

The sequences encoding the chimeric proteins involved in the present invention were as shown below:

58L1DE/16dEs nt
SEQ ID No. 20
ATG TCC GTC TGG CGT CCG AGT GAA GCC ACG GTG TAT TTG CCA CCA GTC CCG GTT AGC AAA GTA
GTG TCC ACC GAC GAG TAC GTG AGC CGC ACT TCG ATC TAC TAC TAC GCA GGA AGC TCG CGC CTG
TTG GCT GTA GGC AAC CCC TAT TTC AGT ATC AAA TCA CCG AAT AAT AAC AAG AAG GTT TTA GTG
CCG AAG GTA TCT GGG CTG CAA TAC CGT GTG TTC CGT GTC CGC CTG CCG GAC CCA AAT AAA TTT
GGA TTT CCT GAT ACT TCC TTC TAT AAT CCT GAC ACC CAA CGT CTG GTA TGG GCC TGT GTT GGA CTT
GAA ATT GGA CGC GGT CAA CCT CTG GGC GTT GGC GTA AGT GGT CAC CCA TAT CTG AAC AAG TTC
GAC GAT ACT GAG ACA TCC AAC CGC TAT TAT AAG ACA TGT AAG CAG GCA GGC ACG TGT CCC CCA
GAT CCT GCT CAG CCC GGG TCG GAC AAC CGT GAA TGC CTT TCA ATG GAC TAC AAG CAA ACC CAA
CTT TGC TTA ATC GGG TGT AAG CCC CCT ACA GGC GAG CAT TGG GGG AAG GGG GTG GCG TGT AAT
AAC AAT GCA GCT GCT ACT GAT TGT CCT CCC CTT GAG CTG TTC AAT AGT ATT ATT GAA GAC GGA
GAC ATG GTA GAT ACT GGG TTT GGC TGT ATG GAC TTT GGG ACG CTT CAA GCG AAT AAG TCA GAT
GTA CCC ATT GAC ATT TGC AAT TCG ACT TGT AAG TAT CCC GAC TAC CTT AAA ATG GCT TCC GAG CCA
TAT GGC GAC AGC CTG TTT TTT TTC CTG CGC CGC GAA CAA ATG TTC GTT CGT CAC TTT TTC AAC CGT
GCT GGG AAA CTT GGC GAG GCT GTC CCG GAC GAC TTA TAC ATT AAG GGG AGC GGA AAC ACA GCT
GTC ATC CAA TCT TCC GCG TTC TTC CCG ACG CCA TCA GGG TCG ATC GTG ACC AGC GAA AGC CAG
CTG TTC AAT AAA CCG TAC TGG TTG CAA CGT GCT CAG GGA CAT AAT AAC GGC ATT TGT TGG GGC
AAC CAG CTT TTC GTA ACA GTA GTA GAC ACG ACT CGT TCA ACT AAC ATG ACG TTG TGT ACG GAG
GTT ACT AAG GAA GGC ACA TAT AAG AAC GAC AAT TTT AAA GAG TAC GTA CGT CAT GTC GAG GAG
TAC GAT CTG CAA TTT GTC TTC CAA CTT TGC AAG ATT ACC CTT ACT GCT GAG ATC ATG ACA TAT ATC
CAT ACA ATG GAT TCT AAT ATT TTG GAA GAC TGG CAG TTC GGC CTT ACT CCC CCT CCT AGC GCG
AGT CTT CAG GAT ACC TAT CGT TTT GTC ACA AGT CAA GCT ATC ACC TGC CAG AAG ACC GCA CCG
CCG AAG GAA AAA GAA GAT CCG CTT AAT AAG TAC ACC TTC TGG GAG GTT AAC TTG AAG GAA AAG
TTC AGC GCC GAC TTG GAT CAA TTT CCC TTA GGT CGT AAA TTC CTT TTG CAG TCT GGA TTG AAA
GCT AAA CCG CGC TTA AAA CGC TCC GCT CCT ACT ACG CGT GCC CCC AGC ACG AAG CGC AAA AAG
GTA AAA AAG TAA
58L1ΔCDE/16dEs nt
SEQ ID No. 21
ATGAGCGTGT GGAGACCCTC CGAAGCAACC GTCTATCTCC CACCCGTCCC CGTCAGCAAA
GTCGTGTCAA CCGACGAGTA CGTCAGCAGG ACCTCAATCT ACTACTACGC TGGTTCCAGT
CGCTTGCTCG CCGTCGGCAA CCCCTACTTC AGTATTAAGT CCCCAAACAA CAACAAGAAG
GTGCTGGTCC CAAAAGTGAG CGGCCTGCAA TACCGCGTGT TCCGCGTCAG GCTGCCCGAC
CCAAACAAGT TCGGCTTCCC CGACACCAGC TTCTACAATC CCGACACCCA GAGGCTCGTG
TGGGCCTGCG TGGGCTTGGA GATCGGCCGC GGCCAACCAC TCGGCGTCGG CGTGTCCGGC
CACCCCTACC TGAACAAGTT CGACGATACC GAGACATCCA ATCGCTATTA CAAGACTTGC
AAGCAGGCTG GTACCTGCCC CCCTGACCCA GCCCAACCCG GCAGCGACAA TCGCGAGTGT
CTGAGCATGG ACTACAAGCA GACCCAGCTG TGCCTGATCG GCTGCAAGCC CCCAACCGGT
GAACACTGGG GCAAGGGCGT CGCTTGCAAC AACAACGCCG CCGCCACCGA CTGCCCACCC
CTCGAGTTGT TCAACAGCAT CATCGAAGAC GGCGATATGG TGGACACCGG CTTCGGCTGT
ATGGATTTCG GCACCCTCCA AGCCAACAAG TCCGACGTCC CCATCGACAT CTGCAATTCC
ACCTGTAAGT ACCCCGACTA CCTGAAGATG GCATCCGAGC CCTACGGCGA CAGCCTCTTC
TTCTTCCTCC GCAGGGAACA AATGTTCGTC CGCCATTTCT TCAACCGCGC CGGCAAGTTG
GGCGAAGCCG TGCCCGACGA TTTGTACATC AAGGGCAGTG GCAACACCGC CGTCATTCAG
TCCTCCGCAT TCTTCCCAAC CCCCTCCGGC AGCATCGTCA CAAGCGAGAG CCAGCTGTTC
AACAAGCCCT ACTGGTTGCA AAGGGCCCAG GGCCACAATA ACGGCATCTG TTGGGGCAAC
CAACTGTTCG TCACAGTCGT CGACACAACC AGGTCAACCA ACATGACCCT GTGTACCGAG
GTGACCAAGG AGGGCACCTA CAAGAACGAC AACTTCAAAG AGTACGTGAG GCACGTCGAG
GAGTACGATC TGCAATTCGT CTTCCAATTG TGTAAGATCA CCTTGACCGC CGAAATCATG
ACCTACATCC ACACCATGGA CAGTAACATC CTCGAAGATT GGCAGTTCGG CCTGACCCCC
CCACCCAGCG CATCCCTGCA AGATACCTAC CGCTTCGTCA CAAGTCAAGC CATCACCTGT
CAGAAGACCG CTCCACCCAA GGAGAAAGAG GACCCCCTGA ACAAGTACAC CTTCTGGGAA
GTCAATCTGA AAGAGAAATT CAGCGCCGAC TTGGACCAAT TCCCACTCGG CAGGAAGTTC
CTGCTGCAGA GCGGCTTGTA A
58L1h4/16dEs nt
SEQ ID No. 22
ATGTCTGTTT GGCGTCCGTC GGAAGCCACC GTTTATCTGC CACCTGTGCC GGTGAGCAAA
GTTGTCTCGA CGGATGAATA TGTGAGTCGC ACCAGCATTT ATTACTATGC AGGTTCTTCG
CGTTTGTTGG CGGTGGGCAA TCCGTACTTT AGCATCAAAT CGCCTAACAA TAACAAAAAG
GTTTTAGTGC CGAAAGTGAG TGGCCTGCAA TATCGTGTGT TTCGCGTCCG CCTGCCGGAT
CCAAATAAGT TTGGGTTCCC TGACACCAGC TTTTATAACC CAGATACCCA GCGTCTGGTT
TGGGCGTGCG TGGGTTTGGA AATTGGCCGC GGCCAACCGT TGGGTGTGGG TGTTTCTGGC
CATCCTTACT TGAATAAATT TGATGACACC GAAACTTCGA ACCGTTATCC GGCCCAGCCA
GGCAGCGACA ATCGGGAATG TCTGTCGATG GATTACAAAC AAACGCAGCT GTGCTTAATT
GGGTGTAAGC CGCCAACCGG TGAGCACTGG GGCAAAGGCG TTGCATGTAA CAATAACGCG
GCGGCCACGG ATTGCCCGCC ATTGGAATTG TTTAATAGTA TCATCGAAGA TGGTGACATG
GTGGATACCG GTTTTGGCTG CATGGATTTC GGCACGTTAC AGGCCAACAA AAGCGACGTG
CCAATTGATA TCTGCAATTC TACCTGTAAG TACCCGGATT ATCTGAAGAT GGCGTCGGAA
CCGTACGGGG ATAGCCTGTT TTTCTTTCTG CGCCGCGAGC AGATGTTCGT TCGTCATTTC
TTTAACCGCG CGGGTAAATT GGGCGAGGCC GTTCCGGACG ATTTGTATAT TAAAGGCTCG
GGGAATACCG CAGTGATTCA GAGTAGCGCG TTTTTCCCTA CCCCATCTGG TTCGATCGTG
ACCAGCGAAT CGCAACTGTT TAACAAGCCG TATTGGCTGC AGCGTGCGCA GGGCCACAAT
AACGGCATCT GCTGGGGTAA TCAACTGTTT GTTACGGTCG TGGACACCAC GCGGAGTACG
AACATGACGC TGTGTACCGA AGTGACTAAA GAGGGTACCT ACAAGAATGA TAACTTTAAA
GAGTATGTTC GCCATGTTGA AGAGTATGAT TTGCAGTTTG TGTTCCAATT GTGCAAAATT
ACGTTAACGG CCGAGATCAT GACTTACATT CACACCATGG ATAGCAATAT TCTGGAAGAC
TGGCAGTTCG GCCTGACGCC GCCACCTTCT GCCTCGCTGC AGGATACCTA TCGCTTTGTG
ACCAGCCAGG CGATCACGTG CCAAAAATAT AAAACCTGCA AACAGGCCGG CACCTGCCCG
CCAGATCCGC CAAAGGAAAA AGAGGACCCG TTGAACAAAT ATACGTTTTG GGAAGTTAAT
TTGAAGGAAA AATTTTCGGC CGATCTGGAC CAGTTCCCAC TGGGCCGTAA ATTTCTGTTA
CAAAGTGGTT TGAAAGCCAA GCCGCGCTTG AAACGTAGCG CGCCAACCAC GCGGGCGCCG
TCTACCAAAC GCAAGAAAGT TAAGAAATAA
58L1ΔCh4/16dEs nt
SEQ ID No. 23
ATGAGCGTGT GGAGACCCTC CGAAGCAACC GTCTATCTCC CACCCGTCCC CGTCAGCAAA
GTCGTGTCAA CCGACGAGTA CGTCAGCAGG ACCTCAATCT ACTACTACGC TGGTTCCAGT
CGCTTGCTCG CCGTCGGCAA CCCCTACTTC AGTATTAAGT CCCCAAACAA CAACAAGAAG
GTGCTGGTCC CAAAAGTGAG CGGCCTGCAA TACCGCGTGT TCCGCGTCAG GCTGCCCGAC
CCAAACAAGT TCGGCTTCCC CGACACCAGC TTCTACAATC CCGACACCCA GAGGCTCGTG
TGGGCCTGCG TGGGCTTGGA GATCGGCCGC GGCCAACCAC TCGGCGTCGG CGTGTCCGGC
CACCCCTACC TGAACAAGTT CGACGATACC GAGACATCCA ATCGCTACCC AGCCCAACCC
GGCAGCGACA ATCGCGAGTG TCTGAGCATG GACTACAAGC AGACCCAGCT GTGCCTGATC
GGCTGCAAGC CCCCAACCGG TGAACACTGG GGCAAGGGCG TCGCTTGCAA CAACAACGCC
GCCGCCACCG ACTGCCCACC CCTCGAGTTG TTCAACAGCA TCATCGAAGA CGGCGATATG
GTGGACACCG GCTTCGGCTG TATGGATTTC GGCACCCTCC AAGCCAACAA GTCCGACGTC
CCCATCGACA TCTGCAATTC CACCTGTAAG TACCCCGACT ACCTGAAGAT GGCATCCGAG
CCCTACGGCG ACAGCCTCTT CTTCTTCCTC CGCAGGGAAC AAATGTTCGT CCGCCATTTC
TTCAACCGCG CCGGCAAGTT GGGCGAAGCC GTGCCCGACG ATTTGTACAT CAAGGGCAGT
GGCAACACCG CCGTCATTCA GTCCTCCGCA TTCTTCCCAA CCCCCTCCGG CAGCATCGTC
ACAAGCGAGA GCCAGCTGTT CAACAAGCCC TACTGGTTGC AAAGGGCCCA GGGCCACAAT
AACGGCATCT GTTGGGGCAA CCAACTGTTC GTCACAGTCG TCGACACAAC CAGGTCAACC
AACATGACCC TGTGTACCGA GGTGACCAAG GAGGGCACCT ACAAGAACGA CAACTTCAAA
GAGTACGTGA GGCACGTCGA GGAGTACGAT CTGCAATTCG TCTTCCAATT GTGTAAGATC
ACCTTGACCG CCGAAATCAT GACCTACATC CACACCATGG ACAGTAACAT CCTCGAAGAT
TGGCAGTTCG GCCTGACCCC CCCACCCAGC GCATCCCTGC AAGATACCTA CCGCTTCGTC
ACAAGTCAAG CCATCACCTG TCAGAAGTAC AAGACCTGCA AGCAGGCCGG TACCTGCCCC
CCTGACCCAC CCAAGGAGAA AGAGGACCCC CTGAACAAGT ACACCTTCTG GGAAGTCAAT
CTGAAAGAGA AATTCAGCGC CGACTTGGAC CAATTCCCAC TCGGCAGGAA GTTCCTGCTG
CAGAGCGGCT TGTAA
58L1ΔN4h4/16dE nt
SEQ ID No. 24
ATGCGTCCGT CGGAAGCCAC CGTTTATCTG CCACCTGTGC CGGTGAGCAA AGTTGTCTCG
ACGGATGAAT ATGTGAGTCG CACCAGCATT TATTACTATG CAGGTTCTTC GCGTTTGTTG
GCGGTGGGCA ATCCGTACTT TAGCATCAAA TCGCCTAACA ATAACAAAAA GGTTTTAGTG
CCGAAAGTGA GTGGCCTGCA ATATCGTGTG TTTCGCGTCC GCCTGCCGGA TCCAAATAAG
TTTGGGTTCC CTGACACCAG CTTTTATAAC CCAGATACCC AGCGTCTGGT TTGGGCGTGC
GTGGGTTTGG AAATTGGCCG CGGCCAACCG TTGGGTGTGG GTGTTTCTGG CCATCCTTAC
TTGAATAAAT TTGATGACAC CGAAACTTCG AACCGTTATC CGGCCCAGCC AGGCAGCGAC
AATCGGGAAT GTCTGTCGAT GGATTACAAA CAAACGCAGC TGTGCTTAAT TGGGTGTAAG
CCGCCAACCG GTGAGCACTG GGGCAAAGGC GTTGCATGTA ACAATAACGC GGCGGCCACG
GATTGCCCGC CATTGGAATT GTTTAATAGT ATCATCGAAG ATGGTGACAT GGTGGATACC
GGTTTTGGCT GCATGGATTT CGGCACGTTA CAGGCCAACA AAAGCGACGT GCCAATTGAT
ATCTGCAATT CTACCTGTAA GTACCCGGAT TATCTGAAGA TGGCGTCGGA ACCGTACGGG
GATAGCCTGT TTTTCTTTCT GCGCCGCGAG CAGATGTTCG TTCGTCATTT CTTTAACCGC
GCGGGTAAAT TGGGCGAGGC CGTTCCGGAC GATTTGTATA TTAAAGGCTC GGGGAATACC
GCAGTGATTC AGAGTAGCGC GTTTTTCCCT ACCCCATCTG GTTCGATCGT GACCAGCGAA
TCGCAACTGT TTAACAAGCC GTATTGGCTG CAGCGTGCGC AGGGCCACAA TAACGGCATC
TGCTGGGGTA ATCAACTGTT TGTTACGGTC GTGGACACCA CGCGGAGTAC GAACATGACG
CTGTGTACCG AAGTGACTAA AGAGGGTACC TACAAGAATG ATAACTTTAA AGAGTATGTT
CGCCATGTTG AAGAGTATGA TTTGCAGTTT GTGTTCCAAT TGTGCAAAAT TACGTTAACG
GCCGAGATCA TGACTTACAT TCACACCATG GATAGCAATA TTCTGGAAGA CTGGCAGTTC
GGCCTGACGC CGCCACCTTC TGCCTCGCTG CAGGATACCT ATCGCTTTGT GACCAGCCAG
GCGATCACGT GCCAAAAACT GTATAAAACC TGCAAACAGG CCGGCACCTG CCCGCCAGAT
ATTATCCCGA AAGTTGAAGG TCCGCCAAAG GAAAAAGAGG ACCCGTTGAA CAAATATACG
TTTTGGGAAG TTAATTTGAA GGAAAAATTT TCGGCCGATC TGGACCAGTT CCCACTGGGC
CGTAAATTTC TGTTACAAAG TGGTTTGAAA GCCAAGCCGC GCTTGAAACG TAGCGCGCCA
ACCACGCGGG CGCCGTCTAC CAAACGCAAG AAAGTTAAGA AATAA
58L1ΔN4Ch4/16dE nt
SEQ ID No. 25
ATGAGACCCT CCGAAGCAAC CGTCTATCTC CCACCCGTCC CCGTCAGCAA AGTCGTGTCA
ACCGACGAGT ACGTCAGCAG GACCTCAATC TACTACTACG CTGGTTCCAG TCGCTTGCTC
GCCGTCGGCA ACCCCTACTT CAGTATTAAG TCCCCAAACA ACAACAAGAA GGTGCTGGTC
CCAAAAGTGA GCGGCCTGCA ATACCGCGTG TTCCGCGTCA GGCTGCCCGA CCCAAACAAG
TTCGGCTTCC CCGACACCAG CTTCTACAAT CCCGACACCC AGAGGCTCGT GTGGGCCTGC
GTGGGCTTGG AGATCGGCCG CGGCCAACCA CTCGGCGTCG GCGTGTCCGG CCACCCCTAC
CTGAACAAGT TCGACGATAC CGAGACATCC AATCGCTACC CAGCCCAACC CGGCAGCGAC
AATCGCGAGT GTCTGAGCAT GGACTACAAG CAGACCCAGC TGTGCCTGAT CGGCTGCAAG
CCCCCAACCG GTGAACACTG GGGCAAGGGC GTCGCTTGCA ACAACAACGC CGCCGCCACC
GACTGCCCAC CCCTCGAGTT GTTCAACAGC ATCATCGAAG ACGGCGATAT GGTGGACACC
GGCTTCGGCT GTATGGATTT CGGCACCCTC CAAGCCAACA AGTCCGACGT CCCCATCGAC
ATCTGCAATT CCACCTGTAA GTACCCCGAC TACCTGAAGA TGGCATCCGA GCCCTACGGC
GACAGCCTCT TCTTCTTCCT CCGCAGGGAA CAAATGTTCG TCCGCCATTT CTTCAACCGC
GCCGGCAAGT TGGGCGAAGC CGTGCCCGAC GATTTGTACA TCAAGGGCAG TGGCAACACC
GCCGTCATTC AGTCCTCCGC ATTCTTCCCA ACCCCCTCCG GCAGCATCGT CACAAGCGAG
AGCCAGCTGT TCAACAAGCC CTACTGGTTG CAAAGGGCCC AGGGCCACAA TAACGGCATC
TGTTGGGGCA ACCAACTGTT CGTCACAGTC GTCGACACAA CCAGGTCAAC CAACATGACC
CTGTGTACCG AGGTGACCAA GGAGGGCACC TACAAGAACG ACAACTTCAA AGAGTACGTG
AGGCACGTCG AGGAGTACGA TCTGCAATTC GTCTTCCAAT TGTGTAAGAT CACCTTGACC
GCCGAAATCA TGACCTACAT CCACACCATG GACAGTAACA TCCTCGAAGA TTGGCAGTTC
GGCCTGACCC CCCCACCCAG CGCATCCCTG CAAGATACCT ACCGCTTCGT CACAAGTCAA
GCCATCACCT GTCAGAAGCT GTACAAGACC TGCAAGCAGG CCGGTACCTG CCCCCCTGAC
ATCATCCCCA AGGTCGAAGG ACCACCCAAG GAGAAAGAGG ACCCCCTGAA CAAGTACACC
TTCTGGGAAG TCAATCTGAA AGAGAAATTC AGCGCCGACT TGGACCAATT CCCACTCGGC
AGGAAGTTCC TGCTGCAGAG CGGCTTGTAA
58L1ΔN4h4/16dEs
SEQ ID No. 26
ATGCGTCCGT CGGAAGCCAC CGTTTATCTG CCACCTGTGC CGGTGAGCAA AGTTGTCTCG
ACGGATGAAT ATGTGAGTCG CACCAGCATT TATTACTATG CAGGTTCTTC GCGTTTGTTG
GCGGTGGGCA ATCCGTACTT TAGCATCAAA TCGCCTAACA ATAACAAAAA GGTTTTAGTG
CCGAAAGTGA GTGGCCTGCA ATATCGTGTG TTTCGCGTCC GCCTGCCGGA TCCAAATAAG
TTTGGGTTCC CTGACACCAG CTTTTATAAC CCAGATACCC AGCGTCTGGT TTGGGCGTGC
GTGGGTTTGG AAATTGGCCG CGGCCAACCG TTGGGTGTGG GTGTTTCTGG CCATCCTTAC
TTGAATAAAT TTGATGACAC CGAAACTTCG AACCGTTATC CGGCCCAGCC AGGCAGCGAC
AATCGGGAAT GTCTGTCGAT GGATTACAAA CAAACGCAGC TGTGCTTAAT TGGGTGTAAG
CCGCCAACCG GTGAGCACTG GGGCAAAGGC GTTGCATGTA ACAATAACGC GGCGGCCACG
GATTGCCCGC CATTGGAATT GTTTAATAGT ATCATCGAAG ATGGTGACAT GGTGGATACC
GGTTTTGGCT GCATGGATTT CGGCACGTTA CAGGCCAACA AAAGCGACGT GCCAATTGAT
ATCTGCAATT CTACCTGTAA GTACCCGGAT TATCTGAAGA TGGCGTCGGA ACCGTACGGG
GATAGCCTGT TTTTCTTTCT GCGCCGCGAG CAGATGTTCG TTCGTCATTT CTTTAACCGC
GCGGGTAAAT TGGGCGAGGC CGTTCCGGAC GATTTGTATA TTAAAGGCTC GGGGAATACC
GCAGTGATTC AGAGTAGCGC GTTTTTCCCT ACCCCATCTG GTTCGATCGT GACCAGCGAA
TCGCAACTGT TTAACAAGCC GTATTGGCTG CAGCGTGCGC AGGGCCACAA TAACGGCATC
TGCTGGGGTA ATCAACTGTT TGTTACGGTC GTGGACACCA CGCGGAGTAC GAACATGACG
CTGTGTACCG AAGTGACTAA AGAGGGTACC TACAAGAATG ATAACTTTAA AGAGTATGTT
CGCCATGTTG AAGAGTATGA TTTGCAGTTT GTGTTCCAAT TGTGCAAAAT TACGTTAACG
GCCGAGATCA TGACTTACAT TCACACCATG GATAGCAATA TTCTGGAAGA CTGGCAGTTC
GGCCTGACGC CGCCACCTTC TGCCTCGCTG CAGGATACCT ATCGCTTTGT GACCAGCCAG
GCGATCACGT GCCAAAAACT GTATAAAACC TGCAAACAGG CCGGCACCTG CCCGCCAGAT
ATTCCGCCAA AGGAAAAAGA GGACCCGTTG AACAAATATA CGTTTTGGGA AGTTAATTTG
AAGGAAAAAT TTTCGGCCGA TCTGGACCAG TTCCCACTGG GCCGTAAATT TCTGTTACAA
AGTGGTTTGA AAGCCAAGCC GCGCTTGAAA CGTAGCGCGC CAACCACGCG GGCGCCGTCT
ACCAAACGCA AGAAAGTTAA GAAATAA
58L1ΔN4Ch4/16dEs nt
SEQ ID No. 27
ATGAGACCCT CCGAAGCAAC CGTCTATCTC CCACCCGTCC CCGTCAGCAA AGTCGTGTCA
ACCGACGAGT ACGTCAGCAG GACCTCAATC TACTACTACG CTGGTTCCAG TCGCTTGCTC
GCCGTCGGCA ACCCCTACTT CAGTATTAAG TCCCCAAACA ACAACAAGAA GGTGCTGGTC
CCAAAAGTGA GCGGCCTGCA ATACCGCGTG TTCCGCGTCA GGCTGCCCGA CCCAAACAAG
TTCGGCTTCC CCGACACCAG CTTCTACAAT CCCGACACCC AGAGGCTCGT GTGGGCCTGC
GTGGGCTTGG AGATCGGCCG CGGCCAACCA CTCGGCGTCG GCGTGTCCGG CCACCCCTAC
CTGAACAAGT TCGACGATAC CGAGACATCC AATCGCTACC CAGCCCAACC CGGCAGCGAC
AATCGCGAGT GTCTGAGCAT GGACTACAAG CAGACCCAGC TGTGCCTGAT CGGCTGCAAG
CCCCCAACCG GTGAACACTG GGGCAAGGGC GTCGCTTGCA ACAACAACGC CGCCGCCACC
GACTGCCCAC CCCTCGAGTT GTTCAACAGC ATCATCGAAG ACGGCGATAT GGTGGACACC
GGCTTCGGCT GTATGGATTT CGGCACCCTC CAAGCCAACA AGTCCGACGT CCCCATCGAC
ATCTGCAATT CCACCTGTAA GTACCCCGAC TACCTGAAGA TGGCATCCGA GCCCTACGGC
GACAGCCTCT TCTTCTTCCT CCGCAGGGAA CAAATGTTCG TCCGCCATTT CTTCAACCGC
GCCGGCAAGT TGGGCGAAGC CGTGCCCGAC GATTTGTACA TCAAGGGCAG TGGCAACACC
GCCGTCATTC AGTCCTCCGC ATTCTTCCCA ACCCCCTCCG GCAGCATCGT CACAAGCGAG
AGCCAGCTGT TCAACAAGCC CTACTGGTTG CAAAGGGCCC AGGGCCACAA TAACGGCATC
TGTTGGGGCA ACCAACTGTT CGTCACAGTC GTCGACACAA CCAGGTCAAC CAACATGACC
CTGTGTACCG AGGTGACCAA GGAGGGCACC TACAAGAACG ACAACTTCAA AGAGTACGTG
AGGCACGTCG AGGAGTACGA TCTGCAATTC GTCTTCCAAT TGTGTAAGAT CACCTTGACC
GCCGAAATCA TGACCTACAT CCACACCATG GACAGTAACA TCCTCGAAGA TTGGCAGTTC
GGCCTGACCC CCCCACCCAG CGCATCCCTG CAAGATACCT ACCGCTTCGT CACAAGTCAA
GCCATCACCT GTCAGAAGCT GTACAAGACT TGCAAGCAGG CTGGTACCTG CCCCCCTGAC
ATCCCACCCA AGGAGAAAGA GGACCCCCTG AACAAGTACA CCTTCTGGGA AGTCAATCTG
AAAGAGAAAT TCAGCGCCGA CTTGGACCAA TTCCCACTCG GCAGGAAGTT CCTGCTGCAG
AGCGGCTTGT AA
58L1ΔN4h4/16dE-CS1 nt
SEQ ID No. 8
ATGAGACCCT CCGAAGCAAC CGTCTATCTC CCACCCGTCC CCGTCAGCAA AGTCGTGTCA
ACCGACGAGT ACGTCAGCAG GACCTCAATC TACTACTACG CTGGTTCCAG TCGCTTGCTC
GCCGTCGGCA ACCCCTACTT CAGTATTAAG TCCCCAAACA ACAACAAGAA GGTGCTGGTC
CCAAAAGTGA GCGGCCTGCA ATACCGCGTG TTCCGCGTCA GGCTGCCCGA CCCAAACAAG
TTCGGCTTCC CCGACACCAG CTTCTACAAT CCCGACACCC AGAGGCTCGT GTGGGCCTGC
GTGGGCTTGG AGATCGGCCG CGGCCAACCA CTCGGCGTCG GCGTGTCCGG CCACCCCTAC
CTGAACAAGT TCGACGATAC CGAGACATCC AATCGCTACC CAGCCCAACC CGGCAGCGAC
AATCGCGAGT GTCTGAGCAT GGACTACAAG CAGACCCAGC TGTGCCTGAT CGGCTGCAAG
CCCCCAACCG GTGAACACTG GGGCAAGGGC GTCGCTTGCA ACAACAACGC CGCCGCCACC
GACTGCCCAC CCCTCGAGTT GTTCAACAGC ATCATCGAAG ACGGCGATAT GGTGGACACC
GGCTTCGGCT GTATGGATTT CGGCACCCTC CAAGCCAACA AGTCCGACGT CCCCATCGAC
ATCTGCAATT CCACCTGTAA GTACCCCGAC TACCTGAAGA TGGCATCCGA GCCCTACGGC
GACAGCCTCT TCTTCTTCCT CCGCAGGGAA CAAATGTTCG TCCGCCATTT CTTCAACCGC
GCCGGCAAGT TGGGCGAAGC CGTGCCCGAC GATTTGTACA TCAAGGGCAG TGGCAACACC
GCCGTCATTC AGTCCTCCGC ATTCTTCCCA ACCCCCTCCG GCAGCATCGT CACAAGCGAG
AGCCAGCTGT TCAACAAGCC CTACTGGTTG CAAAGGGCCC AGGGCCACAA TAACGGCATC
TGTTGGGGCA ACCAACTGTT CGTCACAGTC GTCGACACAA CCAGGTCAAC CAACATGACC
CTGTGTACCG AGGTGACCAA GGAGGGCACC TACAAGAACG ACAACTTCAA AGAGTACGTG
AGGCACGTCG AGGAGTACGA TCTGCAATTC GTCTTCCAAT TGTGTAAGAT CACCTTGACC
GCCGAAATCA TGACCTACAT CCACACCATG GACAGTAACA TCCTCGAAGA TTGGCAGTTC
GGCCTGACCC CCCCACCCAG CGCATCCCTG CAAGATACCT ACCGCTTCGT CACAAGTCAA
GCCATCACCT GTCAGAAGCT GTACAAGACC TGCAAGCAGG CCGGTACCTG CCCCCCTGAC
ATCATCCCCA AGGTCGAAGG ACCACCCAAG GAGAAAGAGG ACCCCCTGAA CAAGTACACC
TTCTGGGAAG TCAATCTGAA AGAGAAATTC AGCGCCGACT TGGACCAATT CCCACTCGGC
AGGAAGTTCC TGCTGCAGAG CGGCTTGAAA GCCGGCCCTT CGTTGGCCGG CTCGGCCCCT
ACGACCTCGG CGCCGTCGAC GGGTGGCAGC GCCGTGGGTA GCTAA
58L1ΔN4h4/16dE-CS2 nt
SEQ ID No. 29
ATGAGACCCT CCGAAGCAAC CGTCTATCTC CCACCCGTCC CCGTCAGCAA AGTCGTGTCA
ACCGACGAGT ACGTCAGCAG GACCTCAATC TACTACTACG CTGGTTCCAG TCGCTTGCTC
GCCGTCGGCA ACCCCTACTT CAGTATTAAG TCCCCAAACA ACAACAAGAA GGTGCTGGTC
CCAAAAGTGA GCGGCCTGCA ATACCGCGTG TTCCGCGTCA GGCTGCCCGA CCCAAACAAG
TTCGGCTTCC CCGACACCAG CTTCTACAAT CCCGACACCC AGAGGCTCGT GTGGGCCTGC
GTGGGCTTGG AGATCGGCCG CGGCCAACCA CTCGGCGTCG GCGTGTCCGG CCACCCCTAC
CTGAACAAGT TCGACGATAC CGAGACATCC AATCGCTACC CAGCCCAACC CGGCAGCGAC
AATCGCGAGT GTCTGAGCAT GGACTACAAG CAGACCCAGC TGTGCCTGAT CGGCTGCAAG
CCCCCAACCG GTGAACACTG GGGCAAGGGC GTCGCTTGCA ACAACAACGC CGCCGCCACC
GACTGCCCAC CCCTCGAGTT GTTCAACAGC ATCATCGAAG ACGGCGATAT GGTGGACACC
GGCTTCGGCT GTATGGATTT CGGCACCCTC CAAGCCAACA AGTCCGACGT CCCCATCGAC
ATCTGCAATT CCACCTGTAA GTACCCCGAC TACCTGAAGA TGGCATCCGA GCCCTACGGC
GACAGCCTCT TCTTCTTCCT CCGCAGGGAA CAAATGTTCG TCCGCCATTT CTTCAACCGC
GCCGGCAAGT TGGGCGAAGC CGTGCCCGAC GATTTGTACA TCAAGGGCAG TGGCAACACC
GCCGTCATTC AGTCCTCCGC ATTCTTCCCA ACCCCCTCCG GCAGCATCGT CACAAGCGAG
AGCCAGCTGT TCAACAAGCC CTACTGGTTG CAAAGGGCCC AGGGCCACAA TAACGGCATC
TGTTGGGGCA ACCAACTGTT CGTCACAGTC GTCGACACAA CCAGGTCAAC CAACATGACC
CTGTGTACCG AGGTGACCAA GGAGGGCACC TACAAGAACG ACAACTTCAA AGAGTACGTG
AGGCACGTCG AGGAGTACGA TCTGCAATTC GTCTTCCAAT TGTGTAAGAT CACCTTGACC
GCCGAAATCA TGACCTACAT CCACACCATG GACAGTAACA TCCTCGAAGA TTGGCAGTTC
GGCCTGACCC CCCCACCCAG CGCATCCCTG CAAGATACCT ACCGCTTCGT CACAAGTCAA
GCCATCACCT GTCAGAAGCT GTACAAGACC TGCAAGCAGG CCGGTACCTG CCCCCCTGAC
ATCATCCCCA AGGTCGAAGG ACCACCCAAG GAGAAAGAGG ACCCCCTGAA CAAGTACACC
TTCTGGGAAG TCAATCTGAA AGAGAAATTC AGCGCCGACT TGGACCAATT CCCACTCGGC
AGGAAGTTCC TGCTGCAGAG CGGCTTGGGT GCCGGCCCTT CGTTGGCCGG CTCGGCCCCT
ACGACCTCGG CGCCGTCGAC GGGTGGCAGC GCCGTGGGTA GCTAA
58L1ΔN4h4/16dE-CS3 nt
SEQ ID No. 30
ATGAGACCCT CCGAAGCAAC CGTCTATCTC CCACCCGTCC CCGTCAGCAA AGTCGTGTCA
ACCGACGAGT ACGTCAGCAG GACCTCAATC TACTACTACG CTGGTTCCAG TCGCTTGCTC
GCCGTCGGCA ACCCCTACTT CAGTATTAAG TCCCCAAACA ACAACAAGAA GGTGCTGGTC
CCAAAAGTGA GCGGCCTGCA ATACCGCGTG TTCCGCGTCA GGCTGCCCGA CCCAAACAAG
TTCGGCTTCC CCGACACCAG CTTCTACAAT CCCGACACCC AGAGGCTCGT GTGGGCCTGC
GTGGGCTTGG AGATCGGCCG CGGCCAACCA CTCGGCGTCG GCGTGTCCGG CCACCCCTAC
CTGAACAAGT TCGACGATAC CGAGACATCC AATCGCTACC CAGCCCAACC CGGCAGCGAC
AATCGCGAGT GTCTGAGCAT GGACTACAAG CAGACCCAGC TGTGCCTGAT CGGCTGCAAG
CCCCCAACCG GTGAACACTG GGGCAAGGGC GTCGCTTGCA ACAACAACGC CGCCGCCACC
GACTGCCCAC CCCTCGAGTT GTTCAACAGC ATCATCGAAG ACGGCGATAT GGTGGACACC
GGCTTCGGCT GTATGGATTT CGGCACCCTC CAAGCCAACA AGTCCGACGT CCCCATCGAC
ATCTGCAATT CCACCTGTAA GTACCCCGAC TACCTGAAGA TGGCATCCGA GCCCTACGGC
GACAGCCTCT TCTTCTTCCT CCGCAGGGAA CAAATGTTCG TCCGCCATTT CTTCAACCGC
GCCGGCAAGT TGGGCGAAGC CGTGCCCGAC GATTTGTACA TCAAGGGCAG TGGCAACACC
GCCGTCATTC AGTCCTCCGC ATTCTTCCCA ACCCCCTCCG GCAGCATCGT CACAAGCGAG
AGCCAGCTGT TCAACAAGCC CTACTGGTTG CAAAGGGCCC AGGGCCACAA TAACGGCATC
TGTTGGGGCA ACCAACTGTT CGTCACAGTC GTCGACACAA CCAGGTCAAC CAACATGACC
CTGTGTACCG AGGTGACCAA GGAGGGCACC TACAAGAACG ACAACTTCAA AGAGTACGTG
AGGCACGTCG AGGAGTACGA TCTGCAATTC GTCTTCCAAT TGTGTAAGAT CACCTTGACC
GCCGAAATCA TGACCTACAT CCACACCATG GACAGTAACA TCCTCGAAGA TTGGCAGTTC
GGCCTGACCC CCCCACCCAG CGCATCCCTG CAAGATACCT ACCGCTTCGT CACAAGTCAA
GCCATCACCT GTCAGAAGCT GTACAAGACC TGCAAGCAGG CCGGTACCTG CCCCCCTGAC
ATCATCCCCA AGGTCGAAGG ACCACCCAAG GAGAAAGAGG ACCCCCTGAA CAAGTACACC
TTCTGGGAAG TCAATCTGAA AGAGAAATTC AGCGCCGACT TGGACCAATT CCCACTCGGC
AGGAAGTTCC TGCTGCAGAG CGGCTTGAAA GCCGGCCCTT CGTTGGCCGG CTCGGCCCCT
ACGACCAGAG CGCCGTCGAC GGGTGGCAGC GCCGTGGGTA GCTAA
58L1h4/16dE
SEQ ID No. 31
ATG AGC GTC TGG CGC CCT TCT GAA GCG ACT GTT TAC CTG CCG CCC GTC CCA GTA AGC AAG GTT
GTG AGC ACC GAT GAA TAT GTT AGT CGT ACA TCG ATC TAT TAC TAT GCT GGA AGC TCA CGT TTG CTG
GCG GTA GGT AAC CCT TAT TTT TCC ATT AAA AGT CCC AAT AAT AAT AAG AAA GTT TTA GTC CCG AAG
GTG TCT GGG TTG CAA TAC CGC GTA TTC CGT GTC CGT CTT CCG GAC CCG AAT AAA TTT GGC TTC
CCC GAT ACT TCT TTT TAT AAT CCC GAT ACT CAG CGT TTA GTA TGG GCC TGT GTT GGG CTT GAG ATC
GGA CGC GGG CAA CCT CTG GGG GTT GGC GTC AGC GGC CAT CCT TAT CTT AAT AAA TTT GAC GAT
ACG GAG ACC AGT AAC CGC TAC CCG GCT CAG CCG GGG TCC GAC AAC CGT GAA TGT TTG TCC ATG
GAT TAT AAG CAG ACT CAG CTG TGT TTG ATC GGT TGT AAA CCT CCT ACA GGT GAA CAT TGG GGC
AAA GGT GTA GCC TGC AAT AAT AAC GCA GCG GCT ACC GAT TGT CCG CCT TTG GAG TTG TTC AAC
TCG ATT ATC GAA GAC GGT GAT ATG GTA GAC ACT GGG TTC GGT TGC ATG GAC TTT GGG ACC CTT
CAA GCC AAT AAG TCC GAC GTG CCA ATC GAC ATT TGC AAC TCT ACA TGC AAG TAC CCT GAC TAT CTT
AAG ATG GCG TCC GAA CCG TAC GGA GAT TCT TTG TTT TTT TTC CTG CGT CGT GAG CAA ATG TTC
GTA CGC CAT TTC TTC AAC CGT GCT GGA AAG TTA GGA GAA GCT GTT CCT GAT GAT CTG TAT ATT
AAG GGA TCT GGG AAC ACA GCG GTT ATT CAA TCC TCC GCT TTC TTT CCA ACG CCA AGC GGG TCC
ATT GTG ACC AGT GAA AGC CAA TTA TTT AAC AAG CCG TAC TGG TTA CAA CGT GCG CAG GGA CAC
AAT AAT GGA ATC TGC TGG GGC AAT CAA TTG TTC GTG ACG GTT GTT GAC ACG ACC CGT TCG ACC
AAT ATG ACC CTT TGC ACT GAG GTA ACT AAG GAG GGT ACG TAC AAA AAT GAC AAT TTT AAG GAG
TAC GTC CGT CAT GTC GAG GAG TAT GAT CTT CAG TTT GTC TTC CAG TTA TGT AAA ATT ACC TTG ACC
GCG GAA ATC ATG ACG TAT ATC CAC ACG ATG GAC TCA AAC ATC CTG GAG GAT TGG CAA TTC GGT
TTG ACT CCG CCC CCG AGC GCG TCT CAA TTG TAT AAG ACT TGT AAG CAG GCA GGT ACA TGC CCA
CCA GAC ATC ATT CCA AAA GTT GAA GGA ACG TGC CAA AAG ACG GCA CCA CCG AAA GAA AAG GAG
GAC CCC CTG AAC AAA TAT ACG TTT TGG GAG GTG AAT TTG AAG GAG AAA TTC TCA GCA GAC TTA
GAC CAG TTT CCG CTT GGC CGC AAG TTT CTG TTG CAG TCT GGG TTG AAG GCA AAA CCG CGT CTT
AAG CGT AGC GCC CCC ACG ACT CGC GCC CCC AGT ACA AAA CGT AAG AAG GTT AAA AAG
58L1ΔCh4/16dE nt
SEQ ID No. 32
ATGAGCGTGT GGAGACCCTC CGAAGCAACC GTCTATCTCC CACCCGTCCC CGTCAGCAAA
GTCGTGTCAA CCGACGAGTA CGTCAGCAGG ACCTCAATCT ACTACTACGC TGGTTCCAGT
CGCTTGCTCG CCGTCGGCAA CCCCTACTTC AGTATTAAGT CCCCAAACAA CAACAAGAAG
GTGCTGGTCC CAAAAGTGAG CGGCCTGCAA TACCGCGTGT TCCGCGTCAG GCTGCCCGAC
CCAAACAAGT TCGGCTTCCC CGACACCAGC TTCTACAATC CCGACACCCA GAGGCTCGTG
TGGGCCTGCG TGGGCTTGGA GATCGGCCGC GGCCAACCAC TCGGCGTCGG CGTGTCCGGC
CACCCCTACC TGAACAAGTT CGACGATACC GAGACATCCA ATCGCTACCC AGCCCAACCC
GGCAGCGACA ATCGCGAGTG TCTGAGCATG GACTACAAGC AGACCCAGCT GTGCCTGATC
GGCTGCAAGC CCCCAACCGG TGAACACTGG GGCAAGGGCG TCGCTTGCAA CAACAACGCC
GCCGCCACCG ACTGCCCACC CCTCGAGTTG TTCAACAGCA TCATCGAAGA CGGCGATATG
GTGGACACCG GCTTCGGCTG TATGGATTTC GGCACCCTCC AAGCCAACAA GTCCGACGTC
CCCATCGACA TCTGCAATTC CACCTGTAAG TACCCCGACT ACCTGAAGAT GGCATCCGAG
CCCTACGGCG ACAGCCTCTT CTTCTTCCTC CGCAGGGAAC AAATGTTCGT CCGCCATTTC
TTCAACCGCG CCGGCAAGTT GGGCGAAGCC GTGCCCGACG ATTTGTACAT CAAGGGCAGT
GGCAACACCG CCGTCATTCA GTCCTCCGCA TTCTTCCCAA CCCCCTCCGG CAGCATCGTC
ACAAGCGAGA GCCAGCTGTT CAACAAGCCC TACTGGTTGC AAAGGGCCCA GGGCCACAAT
AACGGCATCT GTTGGGGCAA CCAACTGTTC GTCACAGTCG TCGACACAAC CAGGTCAACC
AACATGACCC TGTGTACCGA GGTGACCAAG GAGGGCACCT ACAAGAACGA CAACTTCAAA
GAGTACGTGA GGCACGTCGA GGAGTACGAT CTGCAATTCG TCTTCCAATT GTGTAAGATC
ACCTTGACCG CCGAAATCAT GACCTACATC CACACCATGG ACAGTAACAT CCTCGAAGAT
TGGCAGTTCG GCCTGACCCC CCCACCTAGC GCATCCCAAC TGTACAAGAC CTGCAAGCAG
GCCGGTACCT GCCCCCCTGA CATCATCCCC AAGGTCGAAG GCACCTGTCA GAAGACTGCT
CCACCCAAGG AGAAAGAGGA CCCCCTGAAC AAGTACACCT TCTGGGAAGT CAATCTGAAA
GAGAAATTCA GCGCCGACTT GGACCAATTC CCACTCGGCA GGAAGTTCCT GCTGCAGAGC
GGCTTGTAA

### Example 2: Construction of Recombinant Bacmids and Recombinant Baculoviruses of the Chimeric L1 Protein Genes

Recombinant expression vectors comprising the chimeric L1 genes, namely pFastBac1-58L1ΔCDE/16dEs, pFastBac1-58L1ΔCh4/16dEs, pFastBac1-58L1ΔN4Ch4/16dE, pFastBac1-58L1ΔN4Ch4/16dEs, pFastBac1-58L1ΔN4h4/16dE-CS1, pFastBac1-58L1ΔN4h4/16dE-C S2, pFastBac1-58L1ΔN4h4/16dE-C S3, and pFastBac1-58L1ΔCh4/16dE, were used to transform E. coli DH10Bac competent cells, which were screened to obtain recombinant Bacmids. Then the recombinant Bacmids were used to transfect Sf9 insect cells so as to amplify recombinant baculoviruses within the Sf9 cells. Methods of screening of recombinant Bacmids and amplification of recombinant baculoviruses were all well known, for example, the patent CN 101148661 B.

### Example 3: Expression of Genes of Chimeric L1 Proteins in Sf9 Cells

Sf9 cells were inoculated with the 8 types of recombinant baculoviruses of the chimeric L1 genes, respectively, to express the chimeric L1 proteins. After incubation at 27° C. for about 88 h, the fermentation broth was collected and centrifuged at 3,000 rpm for 15 min. The supernatant was discarded, and the cells were washed with PBS for use in expression identification and purification. Methods of infection and expression were publicly available, for example, the patent CN 101148661 B.

### Example 4: Expression of Genes of Chimeric L1 Proteins in E. Coli

Recombinant expression vectors comprising the chimeric L1 genes, namely pET22b-58L1DE/16dEs, pET22b-58L1h4/16dEs, pET22b-58L1ΔN4h4/16dE, pET22b-58L1ΔN4h4/16dEs, and pET22b-58L1h4/16dE, were used to transform E. coli BL21 (DE3).

Single clones were picked and inoculated into 3 ml of LB medium containing ampicillin and incubated at 37° C. overnight. The bacterial fluid cultured overnight was added to LB medium at a ratio of 1:100 and incubated at 37° C. for about 3 h. When the OD600 reached between 0.8-1.0, IPTG was added to a final concentration of 0.5 μM, and the bacterial fluid was incubated at 16° C. for about 12 h and collected.

### Example 5: Identification of the Expression of Chimeric L1 Proteins

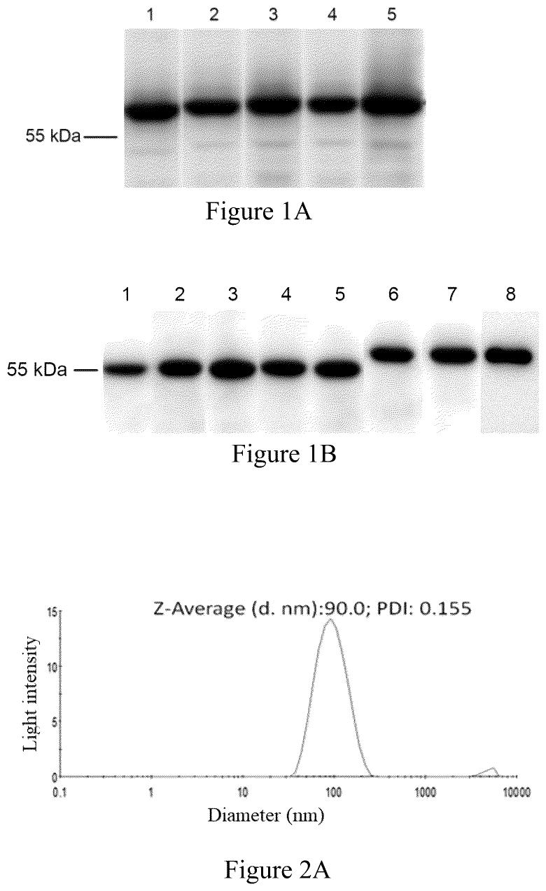

1×10⁶ cells of each of cells expressing the different chimeric L1 proteins described in Examples 3 and 4 were collected and resuspended in 200 μl PBS solution. 50 μl of 6× Loading Buffer was added and the samples were denatured at 75° C. for 8 minutes. 10 μl of sample was used for SDS-PAGE electrophoresis and Western blot identification, respectively. The results were as shown in FIG. 1A to FIG. 1B. All 13 types of chimeric L1 proteins could be expressed at high levels in insect cells or prokaryotic expression systems, among which 58L1DE/16dEs, 58L1h4/16dEs, 58L1ΔN4h4/16dE, 58L1ΔN4h4/16dEs, 58L1h4/16dE, 58L1ΔN4h4/16dE-CS1, 58L1ΔN4h4/16dE-052, and 58L1ΔN4h4/16dE-053 were about 59 kDa in size, while the rest 5 types of proteins were about 55 kDa in size. Methods of SDS-PAGE electrophoresis and Western blot identification were publicly available, for example, the patent CN 101148661 B.

### Example 6: Comparison of the Expression Amounts of Chimeric L1 Proteins in Insect Cells

1×10⁶ cells of each of the cells expressing the wild-type HPV58L1 protein and the 8 types of chimeric L1 proteins described in Example 3 were collected and resuspended in 200 μl PBS solution. The cells were disrupted by ultrasonic disruption (Ningbo Scientz Ultrasonic Cell Disruptor, 2 #probe, 100 W, ultrasound 5 s, interval 7 s, total time 3 min) and centrifuged at a high speed of 12,000 rpm for 10 minutes. The lysed supernatant was collected and the L1 content in the supernatant was detected by sandwich ELISA, which was well known, for example, the patent CN104513826A.

Microtiter plates were coated with HPV58L1 monoclonal antibodies prepared by the inventor at 80 ng/well by incubation at 4° C. overnight. The plate was blocked with 5% BSA-PBST at room temperature for 2 h and then washed for 3 times with PBST. The lysed supernatant was subjected to 2-fold serial dilution with PBS. The HPV58L1 VLP standard was also subjected to serial dilution from a concentration of 2 μg/ml to 0.0625 μg/ml. The diluted samples were added to the plate respectively at 100 μl per well and incubated at 37° C. for 1 h. The plate was washed for 3 times with PBST, and 1:3000 diluted HPV58L1 rabbit polyclonal antibody was added at 100 μl per well and incubated at 37° C. for 1 h. The plate was washed for 3 times with PBST, and 1:3000 diluted HRP-labeled goat anti-mouse IgG (1:3000 dilution, ZSGB-Bio Corporation) was added and incubated at 37° C. for 45 minutes. The plate was washed for 5 times with PBST, and 100 μl of OPD substrate (Sigma) was added to each well for chromogenic reaction at 37° C. for 5 minutes. The reaction was stopped with 50 μl of 2 M sulfuric acid, and the absorbance at 490 nm was determined. The concentrations of the HPV58L1 protein and the 58L1 chimeric proteins in the lysed supernatant were calculated according to the standard curve.

The results were as shown in Table 1. The expression amounts of the HPV58 chimeric L1 proteins of the present invention were all higher than that of the wild-type HPV58L1 backbone. In addition, the expression amounts of the chimeric proteins 58L1ΔN4h4/16dE-CS1, 58L1ΔN4h4/16dE-C S2 and 58L1ΔN4h4/16dE-CS1 with the 58L1 mutant with N-terminus truncation in combination with C-terminus substitution as the backbone were all higher than those of the HPV58L1 backbone and the corresponding C-terminus truncated chimeric protein 58L1ΔN4Ch4/16dE.

TABLE 1
Analysis of expression amounts of chimeric L1 proteins

| | Expression amount (mg/L) | | | |
|---|---|---|---|---|
| Protein name | Batch 1 | Batch 2 | Batch 3 | Average |
| HPV58L1 | 20 | 18 | 15 | 17.7 |
| 58L1ΔCDE/16dEs | 39 | 37 | 33 | 36.3 |
| 58L1ΔCh4/16dEs | 32 | 35 | 30 | 32.3 |
| 58L1ΔN4Ch4/16dE | 53 | 45 | 51 | 49.7 |
| 58L1ΔN4Ch4/16dEs | 55 | 58 | 49 | 54 |
| 58L1ΔCh4/16dE | 89 | 96 | 87 | 90.7 |
| 58L1ΔN4h4/16dE-CS1 | 72 | 76 | 79 | 75.7 |
| 58L1ΔN4h4/16dE-CS2 | 77 | 72 | 71 | 73.3 |
| 58L1ΔN4h4/16dE-CS3 | 69 | 78 | 74 | 73.7 |

### Example 7: Purification and Dynamic Light Scattering Particle Size Analysis of Chimeric L1 Proteins

An appropriate amount of cell fermentation broth of chimeric L1 was collected and the cells were resuspended with 10 ml of PBS. PMSF was added to a final concentration of 1 mg/ml. The cells were ultrasonically disrupted (Ningbo Scientz Ultrasonic Cell Disruptor, 6 #probe, 200 W, ultrasound 5 s, interval 7 s, total time 10 min) and the disrupted supernatant was collected for purification. The purification steps were carried out at room temperature. 4% β-mercaptoethanol (w/w) was added to the lysate to disaggregate VLPs. Then the samples were filtered with 0.22 μm filters, followed by successive purification with DMAE anion exchange chromatography or CM cation exchange chromatography (20 mM Tris, 180 mM NaCl, 4% β-ME, elution at pH 7.9), TMAE anion exchange chromatography or Q cation exchange chromatography (20 mM Tris, 180 mM NaCl, 4% β-ME, elution at pH 7.9) and hydroxyapatite chromatography (100 mM NaH₂PO₄, 30 mM NaCl, 4% (3-ME, elution at pH 6.0). The purified product was concentrated and buffer (20 mM NaH₂PO₄, 500 mM NaCl, pH 6.0) exchange was performed using Planova ultrafiltration system to prompt VLP assembly. The above purification methods were all publicly available, for example, the patents CN 101293918 B, CN 1976718 A, etc.

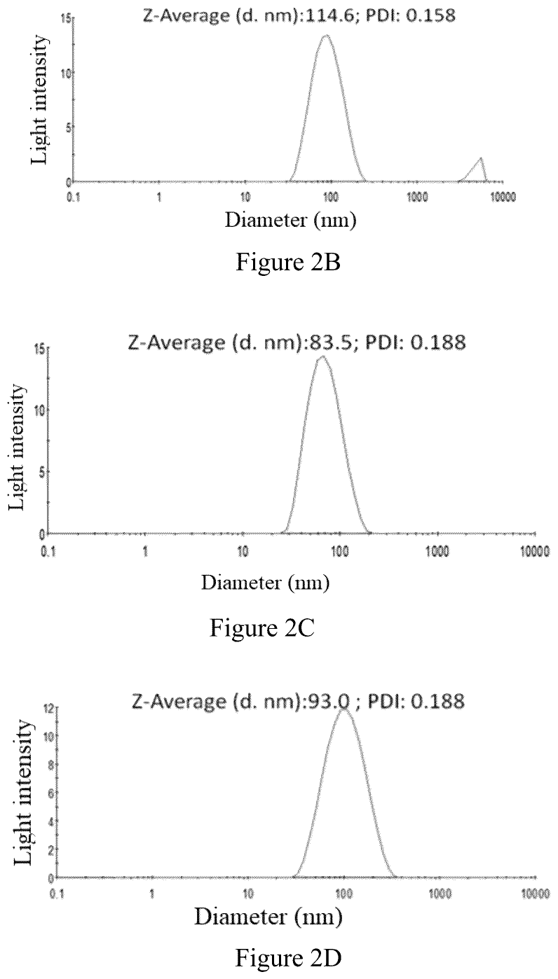

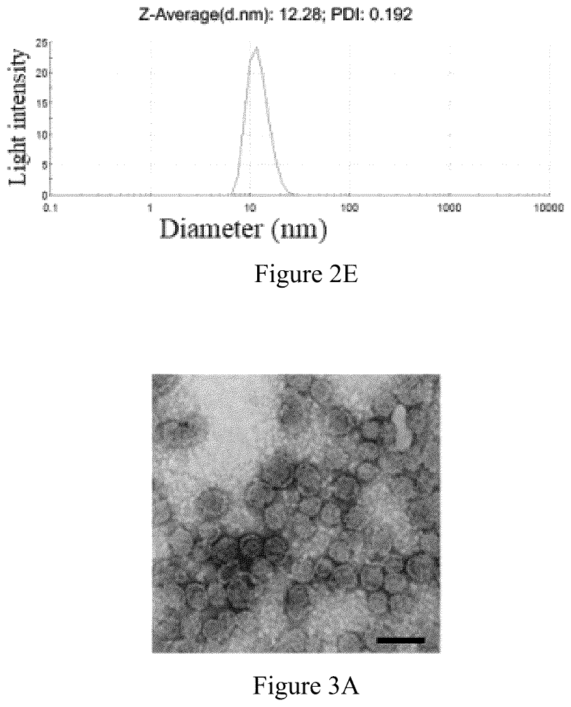

The assembled chimeric protein solutions were subjected to DLS particle size analysis (Zetasizer Nano ZS 90 Dynamic Light Scatterer, Malvern), and the results were as shown in Table 2, wherein the DLS analysis plots of 58L1ΔCDE/16dEs, 58L1ΔCh4/16dEs, 58L1ΔN4Ch4/16dE, 58L1ΔN4Ch4/16dEs and 58L1ΔCh4/16dE were as shown in FIG. 2A to FIG. 2E. The particle sizes of 58L1h4/16dE and 58L1ΔCh4/16dE were only 9.672 nm and 12.28 nm, indicating that the two chimeric proteins were not assembled into VLP.

TABLE 2
DLS analysis of chimeric proteins

| | Hydraulic | |
|---|---|---|
| Chimeric protein name | diameter (nm) | PDI |
| 58L1DE/16dEs | 88 | 0.149 |
| 58L1h4/16dEs | 102.4 | 0.162 |
| 58L1ΔN4h4/16dE | 87.8 | 0.173 |
| 58L1ΔN4h4/16dEs | 95.5 | 0.152 |
| 58L1h4/16dE | 9.672 | 0.185 |
| 58L1ΔCDE/16dE | 90 | 0.155 |
| 58L1ΔCh4/16dEs | 114.6 | 0.158 |
| 58L1ΔN4Ch4/16dE | 83.5 | 0.188 |
| 58L1ΔN4Ch4/16dEs | 93 | 0.188 |
| 58L1ΔCh4/16dE | 12.28 | 0.192 |
| 58L1ΔN4h4/16dE-CS1 | 96.5 | 0.194 |
| 58L1ΔN4h4/16dE-CS2 | 99.2 | 0.187 |
| 58L1ΔN4h4/16dE-CS3 | 102.4 | 0.143 |

### Example 8: Transmission Electron Microscopy Observation of Chimeric VLPs

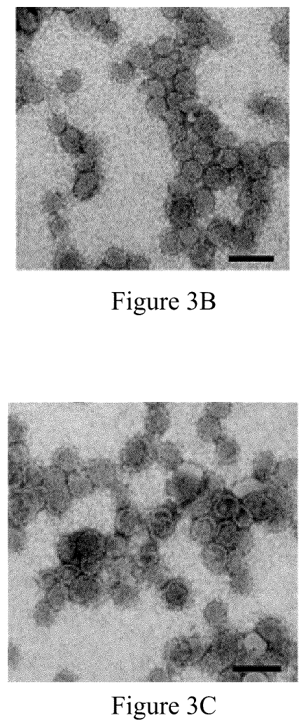

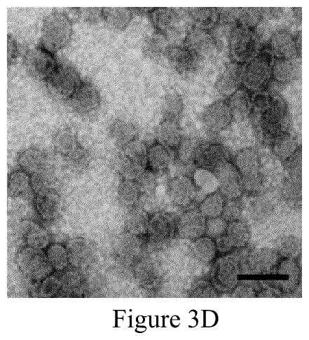

The chimeric proteins were purified separately according to the chromatographic purification method described in Example 7. The assembled chimeras were prepared on copper mesh, stained with 1% uranium acetate, fully dried and then observed using JEM-1400 electron microscope (Olympus). The results showed that 58L1h4/16dE and 58L1ΔCh4/16dE formed chimeric pentamers with a diameter of about 10 nm, and the other chimeric proteins expressed by E. coli and insect cells could all be assembled into chimeric VLPs (cVLPs). The cVLPs expressed by insect cells were about 50 nm in diameter, uniform in size and regular in shape. The prokaryotically expressed cVLPs also had a diameter between 45-50 nm. Part of the results were as shown in FIG. 3A to FIG. 3D. Methods of copper mesh preparation and electron microscopy observation were all publicly available, for example, the patent CN 101148661 B.

### Example 9: Immunization of Mice with Chimeric VLPs and Determination of Neutralizing Antibody Titers

4-6 weeks old BALB/c mice were randomly divided into groups, 5 mice in each group, and 10 μg cVLP, 10 μg HPV58 L1 VLP, 10 μg or 30 μg chimeric pentamers in combination with 50 μg Al(OH)₃ and 5 μg MPL adjuvant were used to immunize the mice by subcutaneous injection at Weeks 0, 4, 7, and 10, for a total of 4 times. Tail vein blood was collected 2 weeks after the 4th immunization and serum was isolated.

15 types of HPV pseudoviruses were used to detect the neutralizing antibody titers of immune serum. The HPV58 neutralizing antibody titer of HPV58L1VLP immune serum was 409600, and no cross-neutralizing antibodies against other types were detected. The HPV58 neutralizing antibody titer of 10 μg 58L1ΔCh4/16dE chimeric pentamer immune serum was 128000, but the cross-neutralization activity was low, and only HPV16 neutralizing antibody was detected (the titer was about 50). The detection results of neutralizing antibodies of cVLPs and 30 μg chimeric pentamers were as shown in Table 3. The level of neutralizing antibodies against the backbone HPV58 induced by 58L1ΔCDE/16dEs cVLP was significantly lower than those of other cVLPs and HPV58L1 VLP, and the level of induced cross-neutralizing antibodies was also very low. After immunizing mice with the rest cVLPs and chimeric pentamers, high levels of HPV58 neutralizing antibody (titer >10⁵) could be induced, which was not statistically different from HPV58L1VLP, and relatively high levels of cross-neutralizing antibodies could be induced. Among them, the immune serum of 58L1ΔCh4/16dEs, 58L1ΔN4Ch4/16dE, 58L1ΔN4Ch4/16dEs, modified cVLPs with C-terminus substitution and 58L1ΔCh4/16dE pentamer not only neutralized HPV58 at high titers, but could also neutralize the rest 14 types of pseudoviruses used for detection. In particular, the immune serum of both 58L1ΔN4Ch4/16dE and 58L1ΔN4Ch4/16dE-CS1 cVLPs neutralized HPV16, -18, -57 pseudoviruses at titers of above 400. It was worth noting that after C-terminus substitution, not only the expression level of 58L1ΔN4Ch4/16dE-CS1 was significantly higher than that of 58L1ΔN4Ch4/16dE, but its immune serum also neutralized the dominant types (HPV16, -18, -57) at increased antibody titers. Methods of pseudovirus preparation and pseudoviral neutralization experiments were all publicly available, for example, the patent CN 104418942A.

Therefore, the cVLPs or chimeric pentamers involved in the present invention can be used as candidates for broad-spectrum HPV vaccines, and can be combined with L1VLPs, cVLPs or chimeric particles of different dominant high-risk types of HPVs to construct broad-spectrum vaccines with relatively low cost, and thus have great value for research and development.

TABLE 3
Neutralizing antibody titers induced by different cVLPs or chimeric pentamers in mice

| | | 58L1ΔCDE/ | 58L1ΔCh4/ | 58L1ΔN4Ch4/ | 58L1ΔN4Ch4/ | 58L1ΔCh4/ | 58L1ΔN4h4/ |
|---|---|---|---|---|---|---|---|
| | | 16dEs | 16dE | 16dE | 16dEs | 16dEs | 16dE-CS1 |
| Average | HPV 58 | 800 | 358667 | 624135 | 563200 | 358667 | 563200 |
| titer | HPV 16 | 50 | 100 | 571 | 165 | 325 | 800 |
| of | HPV 31 | ND* | 15 | 26 | 16 | 25 | 25 |
| neutralizing | HPV 33 | ND | 248 | 297 | 35 | 100 | 440 |
| antibodies | HPV 35 | ND | 37 | 93 | 21 | 125 | 150 |
| | HPV 52 | 50 | 12 | 25 | 22 | 250 | 248 |
| | HPV 18 | 35 | 70 | 489 | 205 | 35 | 600 |
| | HPV 39 | ND | 78 | 104 | 30 | 50 | 78 |
| | HPV 45 | 32 | 56 | 179 | 90 | 100 | 150 |
| | HPV 68 | ND | 28 | 179 | 45 | 50 | 175 |
| | HPV 5 | 32 | 58 | 93 | 35 | 125 | 100 |
| | HPV 27 | ND | 12 | 15 | 16 | 35 | 25 |
| | HPV 57 | 30 | 67 | 414 | 130 | 25 | 530 |
| | HPV 6 | ND | 25 | 54 | 30 | 50 | 50 |
| | HPV 11 | 27 | 39 | 57 | 50 | 50 | 50 |

*ND: Neutralizing antibodies were not detected when serum was 1:10 diluted

1. A human papillomavirus chimeric protein comprising or consisting of a HPV type 58 L1 protein or a mutant of the HPV type 58 L1 protein, and a polypeptide from a HPV type 16 L2 protein inserted into the surface region of the HPV type 58 L1 protein or the mutant of the HPV type 58 L1 protein, wherein the amino acid sequence of the HPV type 58 L1 protein is as shown in SEQ ID No. 1, and the amino acid sequence of the HPV type 16 L2 protein is as shown in SEQ ID No. 2.

2. The human papillomavirus chimeric protein according to claim 1, wherein the amino acid sequence of the human papillomavirus chimeric protein is as shown in any one of SEQ ID Nos. 7-19.

3. A polynucleotide encoding the human papillomavirus chimeric protein according to claim 1 or 2.

4. A vector comprising the polynucleotide according to claim 3.

5. A cell comprising the vector according to claim 4.

6. A polymer which is a chimeric pentamer or chimeric virus-like particle comprising the human papillomavirus chimeric protein according to claim 1, or formed by the human papillomavirus chimeric protein according to claim 1.

7. A method for the prevention of human papillomavirus infection and/or human papillomavirus infection-induced diseases in a subject in need thereof, comprising administering a preventively effective amount of the human papillomavirus chimeric protein according to claim 1 or the polymer according to claim 6 to the subject.

8. A vaccine for the prevention of human papillomavirus infection and/or human papillomavirus infection-induced diseases, comprising the human papillomavirus chimeric protein according to claim 1 or the polymer according to claim 6, an adjuvant, as well as an excipient or carrier for use in vaccines.

9. The vaccine for the prevention of human papillomavirus infection and/or human papillomavirus infection-induced diseases according to claim 8, further comprising at least one virus-like particle or chimeric virus-like particle of mucosa-tropic and/or skin-tropic HIVs.

10. The vaccine for the prevention of papillomavirus infection and/or papillomavirus infection-induced diseases according to claim 8, wherein the adjuvant is an adjuvant for human use.

11. The human papillomavirus chimeric protein according to claim 1, wherein the amino acid sequence of the polypeptide from the HPV type 16 L2 protein is as shown in SEQ ID No. 3, SEQ ID No. 4, SEQ ID No. 5 or SEQ ID No. 6.

12. The human papillomavirus chimeric protein according to claim 1,
wherein the mutant of the HPV type 58 L1 protein comprises any one or more selected from deletion mutation, C-terminus truncation mutation and substitution mutations, compared with the HPV type 58 L1 protein as shown in SEQ ID No. 1,
wherein the deletion mutation is a deletion of amino acids at positions 2-4 at the N-terminus;
the C-terminus truncation mutation is a 25-amino acid truncation at the C-terminus;
the substitution mutations are any group selected from the following groups i) to iii):
i) 476G, 481G, 492G, 493G, 497G, 478S, 487S, 494S, 498S, 480A and 495A;
ii) 474G, 476G, 481G, 492G, 493G, 497G, 478S, 487S, 494S, 498S, 480A and 495A; and
iii) 476G, 481G, 492G, 493G, 497G, 478S, 494S, 498S, 480A and 495A.

13. The human papillomavirus chimeric protein according to claim 1, wherein the polypeptide from the HPV type 16 L2 protein is inserted into the surface region of the HPV type 58 L1 protein or the mutant of the HPV type 58 L1 protein.

14. The human papillomavirus chimeric protein according to claim 1, wherein the polypeptide from the HPV type 16 L2 protein is inserted into the DE loop or h4 region of the HPV type 58 L1 protein or the mutant of the HPV type 58 L1 protein.

15. The human papillomavirus chimeric protein according to claim 1, wherein the polypeptide from the HPV type 16 L2 protein is inserted between amino acids 136 and 137 or between amino acids 431 and 432 of the HPV type 58 L1 protein or the mutant of the HPV type 58 L1 protein by direct insertion, or inserted into the region of amino acids 429 to 432, or the region of amino acids 426 to 429, or the region of amino acids 412 to 426 of the HPV type 58 L1 protein or the mutant of the HPV type 58 L1 protein by non-isometric substitution.

16. The human papillomavirus chimeric protein according to claim 1, wherein the polypeptide from the HPV type 16 L2 protein comprises a linker of 1 to 3 amino acid residues in length at its N-terminus and/or C-terminus.

17. The human papillomavirus chimeric protein according to claim 1, wherein the linker at the N-terminus consists of glycine-proline, and the linker at the C-terminus consists of proline.

18. The polynucleotide according to claim 3, wherein the sequence of the polynucleotide is whole-gene optimized using E. coli codons or whole-gene optimized using insect cell codons.

19. The polynucleotide according to claim 3, wherein the sequence of the polynucleotide is as shown in any one of SEQ ID No. 20 to SEQ ID No. 32.

20. The method according to claim 7, wherein the human papillomavirus infection is an infection selected from one or more of the following human papillomavirus types: HPV16, HPV18, HPV26, HPV31, HPV33, HPV35, HPV39, HPV45, HPV51, HPV52, HPV53, HPV56, HPV58, HPV59, HPV66, HPV68, HPV70, HPV73, HPV6, HPV11, HPV2, HPV5, HPV27 and HPV57; and the human papillomavirus infection-induced diseases are selected from the group consisting of cervical intraepithelial neoplasia, cervical cancer, vulval cancer, penile cancer, vaginal cancer, anal and perianal cancer, oropharyngeal cancer, perianal and genital condylomata acuminata, respiratory recurrent papilloma, skin verrucous hyperplasia, skin squamous cell carcinoma and basal cell carcinoma.


SEQUENCE LISTING (32 sequences)

SEQ ID NO: 1 - protein, 498 aa, HPV 58
MSVWRPSEATVYLPPVPVSKVVSTDEYVSRTSIYYYAGSSRLLAVGNPYFSIKSPNNNKKVLVPKVSGLQYRVFRVRLPDPNKFGFPDTSFYNPDTQRLVWACVGLEIGRGQPLGVGVSGHPYLNKFDDTETSNRYPAQPGSDNRECLSMDYKQTQLCLIGCKPPTGEHWGKGVACNNNAAATDCPPLELFNSIIEDGDMVDTGFGCMDFGTLQANKSDVPIDICNSTCKYPDYLKMASEPYGDSLFFFLRREQMFVRHFFNRAGKLGEAVPDDLYIKGSGNTAVIQSSAFFPTPSGSIVTSESQLFNKPYWLQRAQGHNNGICWGNQLFVTVVDTTRSTNMTLCTEVTKEGTYKNDNFKEYVRHVEEYDLQFVFQLCKITLTAEIMTYIHTMDSNILEDWQFGLTPPPSASLQDTYRFVTSQAITCQKTAPPKEKEDPLNKYTFWEVNLKEKFSADLDQFPLGRKFLLQSGLKAKPRLKRSAPTTRAPSTKRKKVKK

SEQ ID NO: 2 - protein, 473 aa, HPV 16
MRHKRSAKRTKRASATQLYKTCKQAGTCPPDIIPKVEGKTIAEQILQYGSMGVFFGGLGIGTGSGTGGRTGYIPLGTRPPTATDTLAPVRPPLTVDPVGPSDPSIVSLVEETSFIDAGAPTSVPSIPPDVSGFSITTSTDTTPAILDINNTVTTVTTHNNPTFTDPSVLQPPTPAETGGHFTLSSSTISTHNYEEIPMDTFIVSTNPNTVTSSTPIPGSRPVARLGLYSRTTQQVKVVDPAFVTTPTKLITYDNPAYEGIDVDNTLYFSSNDNSINIAPDPDFLDIVALHRPALTSRRTGIRYSRIGNKQTLRTRSGKSIGAKVHYYYDLSTIDPAEEIELQTITPSTYTTTSHAASPTSINNGLYDIYADDFITDTSTTPVPSVPSTSLSGYIPANTTIPFGGAYNIPLVSGPDIPINITDQAPSLIPIVPGSPQYTIIADAGDFYLHPSYYMLRKRRKRLPYFFSDVSLAA

SEQ ID NO: 3 - protein, 22 aa, HPV 16
QLYKTCKQAGTCPPDIIPKVEG

SEQ ID NO: 4 - protein, 21 aa, HPV 16
LYKTCKQAGTCPPDIIPKVEG

SEQ ID NO: 5 - protein, 15 aa, HPV 16
LYKTCKQAGTCPPDI

SEQ ID NO: 6 - protein, 13 aa, HPV 16
YKTCKQAGTCPPD

SEQ ID NO: 7 - protein, 511 aa, Artificial Sequence
  PEPTIDE (1)..(511): Synthetic 58L1DE/16dEs
MSVWRPSEATVYLPPVPVSKVVSTDEYVSRTSIYYYAGSSRLLAVGNPYFSIKSPNNNKKVLVPKVSGLQYRVFRVRLPDPNKFGFPDTSFYNPDTQRLVWACVGLEIGRGQPLGVGVSGHPYLNKFDDTETSNRYYKTCKQAGTCPPDPAQPGSDNRECLSMDYKQTQLCLIGCKPPTGEHWGKGVACNNNAAATDCPPLELFNSIIEDGDMVDTGFGCMDFGTLQANKSDVPIDICNSTCKYPDYLKMASEPYGDSLFFFLRREQMFVRHFFNRAGKLGEAVPDDLYIKGSGNTAVIQSSAFFPTPSGSIVTSESQLFNKPYWLQRAQGHNNGICWGNQLFVTVVDTTRSTNMTLCTEVTKEGTYKNDNFKEYVRHVEEYDLQFVFQLCKITLTAEIMTYIHTMDSNILEDWQFGLTPPPSASLQDTYRFVTSQAITCQKTAPPKEKEDPLNKYTFWEVNLKEKFSADLDQFPLGRKFLLQSGLKAKPRLKRSAPTTRAPSTKRKKVKK

SEQ ID NO: 8 - protein, 486 aa, Artificial Sequence
  PEPTIDE (1)..(484): Synthetic 58L1 delta CDE/16dEs
MSVWRPSEATVYLPPVPVSKVVSTDEYVSRTSIYYYAGSSRLLAVGNPYFSIKSPNNNKKVLVPKVSGLQYRVFRVRLPDPNKFGFPDTSFYNPDTQRLVWACVGLEIGRGQPLGVGVSGHPYLNKFDDTETSNRYYKTCKQAGTCPPDPAQPGSDNRECLSMDYKQTQLCLIGCKPPTGEHWGKGVACNNNAAATDCPPLELFNSIIEDGDMVDTGFGCMDFGTLQANKSDVPIDICNSTCKYPDYLKMASEPYGDSLFFFLRREQMFVRHFFNRAGKLGEAVPDDLYIKGSGNTAVIQSSAFFPTPSGSIVTSESQLFNKPYWLQRAQGHNNGICWGNQLFVTVVDTTRSTNMTLCTEVTKEGTYKNDNFKEYVRHVEEYDLQFVFQLCKITLTAEIMTYIHTMDSNILEDWQFGLTPPPSASLQDTYRFVTSQAITCQKTAPPKEKEDPLNKYTFWEVNLKEKFSADLDQFPLGRKFLLQSGL

SEQ ID NO: 9 - protein, 509 aa, Artificial Sequence
  PEPTIDE (1)..(509): Synthetic 58L1h4/16dEs
MSVWRPSEATVYLPPVPVSKVVSTDEYVSRTSIYYYAGSSRLLAVGNPYFSIKSPNNNKKVLVPKVSGLQYRVFRVRLPDPNKFGFPDTSFYNPDTQRLVWACVGLEIGRGQPLGVGVSGHPYLNKFDDTETSNRYPAQPGSDNRECLSMDYKQTQLCLIGCKPPTGEHWGKGVACNNNAAATDCPPLELFNSIIEDGDMVDTGFGCMDFGTLQANKSDVPIDICNSTCKYPDYLKMASEPYGDSLFFFLRREQMFVRHFFNRAGKLGEAVPDDLYIKGSGNTAVIQSSAFFPTPSGSIVTSESQLFNKPYWLQRAQGHNNGICWGNQLFVTVVDTTRSTNMTLCTEVTKEGTYKNDNFKEYVRHVEEYDLQFVFQLCKITLTAEIMTYIHTMDSNILEDWQFGLTPPPSASLQDTYRFVTSQAITCQKYKTCKQAGTCPPDPPKEKEDPLNKYTFWEVNLKEKFSADLDQFPLGRKFLLQSGLKAKPRLKRSAPTTRAPSTKRKKVKK

SEQ ID NO: 10 - protein, 484 aa, Artificial Sequence
  PEPTIDE (1)..(484): Synthetic 58L1 delta Ch4/16dEs
MSVWRPSEATVYLPPVPVSKVVSTDEYVSRTSIYYYAGSSRLLAVGNPYFSIKSPNNNKKVLVPKVSGLQYRVFRVRLPDPNKFGFPDTSFYNPDTQRLVWACVGLEIGRGQPLGVGVSGHPYLNKFDDTETSNRYPAQPGSDNRECLSMDYKQTQLCLIGCKPPTGEHWGKGVACNNNAAATDCPPLELFNSIIEDGDMVDTGFGCMDFGTLQANKSDVPIDICNSTCKYPDYLKMASEPYGDSLFFFLRREQMFVRHFFNRAGKLGEAVPDDLYIKGSGNTAVIQSSAFFPTPSGSIVTSESQLFNKPYWLQRAQGHNNGICWGNQLFVTVVDTTRSTNMTLCTEVTKEGTYKNDNFKEYVRHVEEYDLQFVFQLCKITLTAEIMTYIHTMDSNILEDWQFGLTPPPSASLQDTYRFVTSQAITCQKYKTCKQAGTCPPDPPKEKEDPLNKYTFWEVNLKEKFSADLDQFPLGRKFLLQSGL

SEQ ID NO: 11 - protein, 514 aa, Artificial Sequence
  PEPTIDE (1)..(514): Synthetic 58L1 delta N4h4/16dE
MRPSEATVYLPPVPVSKVVSTDEYVSRTSIYYYAGSSRLLAVGNPYFSIKSPNNNKKVLVPKVSGLQYRVFRVRLPDPNKFGFPDTSFYNPDTQRLVWACVGLEIGRGQPLGVGVSGHPYLNKFDDTETSNRYPAQPGSDNRECLSMDYKQTQLCLIGCKPPTGEHWGKGVACNNNAAATDCPPLELFNSIIEDGDMVDTGFGCMDFGTLQANKSDVPIDICNSTCKYPDYLKMASEPYGDSLFFFLRREQMFVRHFFNRAGKLGEAVPDDLYIKGSGNTAVIQSSAFFPTPSGSIVTSESQLFNKPYWLQRAQGHNNGICWGNQLFVTVVDTTRSTNMTLCTEVTKEGTYKNDNFKEYVRHVEEYDLQFVFQLCKITLTAEIMTYIHTMDSNILEDWQFGLTPPPSASLQDTYRFVTSQAITCQKLYKTCKQAGTCPPDIIPKVEGPPKEKEDPLNKYTFWEVNLKEKFSADLDQFPLGRKFLLQSGLKAKPRLKRSAPTTRAPSTKRKKVKK

SEQ ID NO: 12 - protein, 489 aa, Artificial Sequence
  PEPTIDE (1)..(489): Synthetic 58L1 delta N4Ch4/16dE
MRPSEATVYLPPVPVSKVVSTDEYVSRTSIYYYAGSSRLLAVGNPYFSIKSPNNNKKVLVPKVSGLQYRVFRVRLPDPNKFGFPDTSFYNPDTQRLVWACVGLEIGRGQPLGVGVSGHPYLNKFDDTETSNRYPAQPGSDNRECLSMDYKQTQLCLIGCKPPTGEHWGKGVACNNNAAATDCPPLELFNSIIEDGDMVDTGFGCMDFGTLQANKSDVPIDICNSTCKYPDYLKMASEPYGDSLFFFLRREQMFVRHFFNRAGKLGEAVPDDLYIKGSGNTAVIQSSAFFPTPSGSIVTSESQLFNKPYWLQRAQGHNNGICWGNQLFVTVVDTTRSTNMTLCTEVTKEGTYKNDNFKEYVRHVEEYDLQFVFQLCKITLTAEIMTYIHTMDSNILEDWQFGLTPPPSASLQDTYRFVTSQAITCQKLYKTCKQAGTCPPDIIPKVEGPPKEKEDPLNKYTFWEVNLKEKFSADLDQFPLGRKFLLQSGL

SEQ ID NO: 13 - protein, 508 aa, Artificial Sequence
  PEPTIDE (1)..(508): Synthetic 58L1 delta N4h4/16dEs
MRPSEATVYLPPVPVSKVVSTDEYVSRTSIYYYAGSSRLLAVGNPYFSIKSPNNNKKVLVPKVSGLQYRVFRVRLPDPNKFGFPDTSFYNPDTQRLVWACVGLEIGRGQPLGVGVSGHPYLNKFDDTETSNRYPAQPGSDNRECLSMDYKQTQLCLIGCKPPTGEHWGKGVACNNNAAATDCPPLELFNSIIEDGDMVDTGFGCMDFGTLQANKSDVPIDICNSTCKYPDYLKMASEPYGDSLFFFLRREQMFVRHFFNRAGKLGEAVPDDLYIKGSGNTAVIQSSAFFPTPSGSIVTSESQLFNKPYWLQRAQGHNNGICWGNQLFVTVVDTTRSTNMTLCTEVTKEGTYKNDNFKEYVRHVEEYDLQFVFQLCKITLTAEIMTYIHTMDSNILEDWQFGLTPPPSASLQDTYRFVTSQAITCQKLYKTCKQAGTCPPDIPPKEKEDPLNKYTFWEVNLKEKFSADLDQFPLGRKFLLQSGLKAKPRLKRSAPTTRAPSTKRKKVKK

SEQ ID NO: 14 - protein, 483 aa, Artificial Sequence
  PEPTIDE (1)..(483): Synthetic 58L1 delta N4Ch4/16dEs
MRPSEATVYLPPVPVSKVVSTDEYVSRTSIYYYAGSSRLLAVGNPYFSIKSPNNNKKVLVPKVSGLQYRVFRVRLPDPNKFGFPDTSFYNPDTQRLVWACVGLEIGRGQPLGVGVSGHPYLNKFDDTETSNRYPAQPGSDNRECLSMDYKQTQLCLIGCKPPTGEHWGKGVACNNNAAATDCPPLELFNSIIEDGDMVDTGFGCMDFGTLQANKSDVPIDICNSTCKYPDYLKMASEPYGDSLFFFLRREQMFVRHFFNRAGKLGEAVPDDLYIKGSGNTAVIQSSAFFPTPSGSIVTSESQLFNKPYWLQRAQGHNNGICWGNQLFVTVVDTTRSTNMTLCTEVTKEGTYKNDNFKEYVRHVEEYDLQFVFQLCKITLTAEIMTYIHTMDSNILEDWQFGLTPPPSASLQDTYRFVTSQAITCQKLYKTCKQAGTCPPDIPPKEKEDPLNKYTFWEVNLKEKFSADLDQFPLGRKFLLQSGL

SEQ ID NO: 15 - protein, 514 aa, Artificial Sequence
  PEPTIDE (1)..(514): Synthetic 58L1 delta N4h4/16dE-CS1
MRPSEATVYLPPVPVSKVVSTDEYVSRTSIYYYAGSSRLLAVGNPYFSIKSPNNNKKVLVPKVSGLQYRVFRVRLPDPNKFGFPDTSFYNPDTQRLVWACVGLEIGRGQPLGVGVSGHPYLNKFDDTETSNRYPAQPGSDNRECLSMDYKQTQLCLIGCKPPTGEHWGKGVACNNNAAATDCPPLELFNSIIEDGDMVDTGFGCMDFGTLQANKSDVPIDICNSTCKYPDYLKMASEPYGDSLFFFLRREQMFVRHFFNRAGKLGEAVPDDLYIKGSGNTAVIQSSAFFPTPSGSIVTSESQLFNKPYWLQRAQGHNNGICWGNQLFVTVVDTTRSTNMTLCTEVTKEGTYKNDNFKEYVRHVEEYDLQFVFQLCKITLTAEIMTYIHTMDSNILEDWQFGLTPPPSASLQDTYRFVTSQAITCQKLYKTCKQAGTCPPDIIPKVEGPPKEKEDPLNKYTFWEVNLKEKFSADLDQFPLGRKFLLQSGLKAGPSLAGSAPTTSAPSTGGSAVGS

SEQ ID NO: 16 - protein, 514 aa, Artificial Sequence
  PEPTIDE (1)..(514): Synthetic 58L1 delta N4h4/16dE-CS2
MRPSEATVYLPPVPVSKVVSTDEYVSRTSIYYYAGSSRLLAVGNPYFSIKSPNNNKKVLVPKVSGLQYRVFRVRLPDPNKFGFPDTSFYNPDTQRLVWACVGLEIGRGQPLGVGVSGHPYLNKFDDTETSNRYPAQPGSDNRECLSMDYKQTQLCLIGCKPPTGEHWGKGVACNNNAAATDCPPLELFNSIIEDGDMVDTGFGCMDFGTLQANKSDVPIDICNSTCKYPDYLKMASEPYGDSLFFFLRREQMFVRHFFNRAGKLGEAVPDDLYIKGSGNTAVIQSSAFFPTPSGSIVTSESQLFNKPYWLQRAQGHNNGICWGNQLFVTVVDTTRSTNMTLCTEVTKEGTYKNDNFKEYVRHVEEYDLQFVFQLCKITLTAEIMTYIHTMDSNILEDWQFGLTPPPSASLQDTYRFVTSQAITCQKLYKTCKQAGTCPPDIIPKVEGPPKEKEDPLNKYTFWEVNLKEKFSADLDQFPLGRKFLLQSGLGAGPSLAGSAPTTSAPSTGGSAVGS

SEQ ID NO: 17 - protein, 514 aa, Artificial Sequence
  PEPTIDE (1)..(514): Synthetic 58L1 delta N4h4/16dE-CS3
MRPSEATVYLPPVPVSKVVSTDEYVSRTSIYYYAGSSRLLAVGNPYFSIKSPNNNKKVLVPKVSGLQYRVFRVRLPDPNKFGFPDTSFYNPDTQRLVWACVGLEIGRGQPLGVGVSGHPYLNKFDDTETSNRYPAQPGSDNRECLSMDYKQTQLCLIGCKPPTGEHWGKGVACNNNAAATDCPPLELFNSIIEDGDMVDTGFGCMDFGTLQANKSDVPIDICNSTCKYPDYLKMASEPYGDSLFFFLRREQMFVRHFFNRAGKLGEAVPDDLYIKGSGNTAVIQSSAFFPTPSGSIVTSESQLFNKPYWLQRAQGHNNGICWGNQLFVTVVDTTRSTNMTLCTEVTKEGTYKNDNFKEYVRHVEEYDLQFVFQLCKITLTAEIMTYIHTMDSNILEDWQFGLTPPPSASLQDTYRFVTSQAITCQKLYKTCKQAGTCPPDIIPKVEGPPKEKEDPLNKYTFWEVNLKEKFSADLDQFPLGRKFLLQSGLKAGPSLAGSAPTTRAPSTGGSAVGS

SEQ ID NO: 18 - protein, 507 aa, Artificial Sequence
  PEPTIDE (1)..(507): Synthetic 58L1h4/16dE
MSVWRPSEATVYLPPVPVSKVVSTDEYVSRTSIYYYAGSSRLLAVGNPYFSIKSPNNNKKVLVPKVSGLQYRVFRVRLPDPNKFGFPDTSFYNPDTQRLVWACVGLEIGRGQPLGVGVSGHPYLNKFDDTETSNRYPAQPGSDNRECLSMDYKQTQLCLIGCKPPTGEHWGKGVACNNNAAATDCPPLELFNSIIEDGDMVDTGFGCMDFGTLQANKSDVPIDICNSTCKYPDYLKMASEPYGDSLFFFLRREQMFVRHFFNRAGKLGEAVPDDLYIKGSGNTAVIQSSAFFPTPSGSIVTSESQLFNKPYWLQRAQGHNNGICWGNQLFVTVVDTTRSTNMTLCTEVTKEGTYKNDNFKEYVRHVEEYDLQFVFQLCKITLTAEIMTYIHTMDSNILEDWQFGLTPPPSASQLYKTCKQAGTCPPDIIPKVEGTCQKTAPPKEKEDPLNKYTFWEVNLKEKFSADLDQFPLGRKFLLQSGLKAKPRLKRSAPTTRAPSTKRKKVKK

SEQ ID NO: 19 - protein, 482 aa, Artificial Sequence
  PEPTIDE (1)..(482): Synthetic 58L1 delta Ch4/16dE
MSVWRPSEATVYLPPVPVSKVVSTDEYVSRTSIYYYAGSSRLLAVGNPYFSIKSPNNNKKVLVPKVSGLQYRVFRVRLPDPNKFGFPDTSFYNPDTQRLVWACVGLEIGRGQPLGVGVSGHPYLNKFDDTETSNRYPAQPGSDNRECLSMDYKQTQLCLIGCKPPTGEHWGKGVACNNNAAATDCPPLELFNSIIEDGDMVDTGFGCMDFGTLQANKSDVPIDICNSTCKYPDYLKMASEPYGDSLFFFLRREQMFVRHFFNRAGKLGEAVPDDLYIKGSGNTAVIQSSAFFPTPSGSIVTSESQLFNKPYWLQRAQGHNNGICWGNQLFVTVVDTTRSTNMTLCTEVTKEGTYKNDNFKEYVRHVEEYDLQFVFQLCKITLTAEIMTYIHTMDSNILEDWQFGLTPPPSASQLYKTCKQAGTCPPDIIPKVEGTCQKTAPPKEKEDPLNKYTFWEVNLKEKFSADLDQFPLGRKFLLQSGL

SEQ ID NO: 20 - DNA, 1536 bp, Artificial Sequence
  gene (1)..(1536): Synthetic 58L1DE/16dEs nt
atgtccgtctggcgtccgagtgaagccacggtgtatttgccaccagtcccggttagcaaagtagtgtccaccgacgagtacgtgagccgcacttcgatctactactacgcaggaagctcgcgcctgttggctgtaggcaacccctatttcagtatcaaatcaccgaataataacaagaaggttttagtgccgaaggtatctgggctgcaataccgtgtgttccgtgtccgcctgccggacccaaataaatttggatttcctgatacttccttctataatcctgacacccaacgtctggtatgggcctgtgttggacttgaaattggacgcggtcaacctctgggcgttggcgtaagtggtcacccatatctgaacaagttcgacgatactgagacatccaaccgctattataagacatgtaagcaggcaggcacgtgtcccccagatcctgctcagcccgggtcggacaaccgtgaatgcctttcaatggactacaagcaaacccaactttgcttaatcgggtgtaagccccctacaggcgagcattgggggaagggggtggcgtgtaataacaatgcagctgctactgattgtcctccccttgagctgttcaatagtattattgaagacggagacatggtagatactgggtttggctgtatggactttgggacgcttcaagcgaataagtcagatgtacccattgacatttgcaattcgacttgtaagtatcccgactaccttaaaatggcttccgagccatatggcgacagcctgttttttttcctgcgccgcgaacaaatgttcgttcgtcactttttcaaccgtgctgggaaacttggcgaggctgtcccggacgacttatacattaaggggagcggaaacacagctgtcatccaatcttccgcgttcttcccgacgccatcagggtcgatcgtgaccagcgaaagccagctgttcaataaaccgtactggttgcaacgtgctcagggacataataacggcatttgttggggcaaccagcttttcgtaacagtagtagacacgactcgttcaactaacatgacgttgtgtacggaggttactaaggaaggcacatataagaacgacaattttaaagagtacgtacgtcatgtcgaggagtacgatctgcaatttgtcttccaactttgcaagattacccttactgctgagatcatgacatatatccatacaatggattctaatattttggaagactggcagttcggccttactccccctcctagcgcgagtcttcaggatacctatcgttttgtcacaagtcaagctatcacctgccagaagaccgcaccgccgaaggaaaaagaagatccgcttaataagtacaccttctgggaggttaacttgaaggaaaagttcagcgccgacttggatcaatttcccttaggtcgtaaattccttttgcagtctggattgaaagctaaaccgcgcttaaaacgctccgctcctactacgcgtgcccccagcacgaagcgcaaaaaggtaaaaaagtaa

SEQ ID NO: 21 - DNA, 1461 bp, Artificial Sequence
  gene (1)..(1461): Synthetic 58L1 delta CDE/16dEs nt
atgagcgtgtggagaccctccgaagcaaccgtctatctcccacccgtccccgtcagcaaagtcgtgtcaaccgacgagtacgtcagcaggacctcaatctactactacgctggttccagtcgcttgctcgccgtcggcaacccctacttcagtattaagtccccaaacaacaacaagaaggtgctggtcccaaaagtgagcggcctgcaataccgcgtgttccgcgtcaggctgcccgacccaaacaagttcggcttccccgacaccagcttctacaatcccgacacccagaggctcgtgtgggcctgcgtgggcttggagatcggccgcggccaaccactcggcgtcggcgtgtccggccacccctacctgaacaagttcgacgataccgagacatccaatcgctattacaagacttgcaagcaggctggtacctgcccccctgacccagcccaacccggcagcgacaatcgcgagtgtctgagcatggactacaagcagacccagctgtgcctgatcggctgcaagcccccaaccggtgaacactggggcaagggcgtcgcttgcaacaacaacgccgccgccaccgactgcccacccctcgagttgttcaacagcatcatcgaagacggcgatatggtggacaccggcttcggctgtatggatttcggcaccctccaagccaacaagtccgacgtccccatcgacatctgcaattccacctgtaagtaccccgactacctgaagatggcatccgagccctacggcgacagcctcttcttcttcctccgcagggaacaaatgttcgtccgccatttcttcaaccgcgccggcaagttgggcgaagccgtgcccgacgatttgtacatcaagggcagtggcaacaccgccgtcattcagtcctccgcattcttcccaaccccctccggcagcatcgtcacaagcgagagccagctgttcaacaagccctactggttgcaaagggcccagggccacaataacggcatctgttggggcaaccaactgttcgtcacagtcgtcgacacaaccaggtcaaccaacatgaccctgtgtaccgaggtgaccaaggagggcacctacaagaacgacaacttcaaagagtacgtgaggcacgtcgaggagtacgatctgcaattcgtcttccaattgtgtaagatcaccttgaccgccgaaatcatgacctacatccacaccatggacagtaacatcctcgaagattggcagttcggcctgacccccccacccagcgcatccctgcaagatacctaccgcttcgtcacaagtcaagccatcacctgtcagaagaccgctccacccaaggagaaagaggaccccctgaacaagtacaccttctgggaagtcaatctgaaagagaaattcagcgccgacttggaccaattcccactcggcaggaagttcctgctgcagagcggcttgtaa

SEQ ID NO: 22 - DNA, 1530 bp, Artificial Sequence
  gene (1)..(1530): Synthetic 58L1h4/16dEs nt
atgtctgtttggcgtccgtcggaagccaccgtttatctgccacctgtgccggtgagcaaagttgtctcgacggatgaatatgtgagtcgcaccagcatttattactatgcaggttcttcgcgtttgttggcggtgggcaatccgtactttagcatcaaatcgcctaacaataacaaaaaggttttagtgccgaaagtgagtggcctgcaatatcgtgtgtttcgcgtccgcctgccggatccaaataagtttgggttccctgacaccagcttttataacccagatacccagcgtctggtttgggcgtgcgtgggtttggaaattggccgcggccaaccgttgggtgtgggtgtttctggccatccttacttgaataaatttgatgacaccgaaacttcgaaccgttatccggcccagccaggcagcgacaatcgggaatgtctgtcgatggattacaaacaaacgcagctgtgcttaattgggtgtaagccgccaaccggtgagcactggggcaaaggcgttgcatgtaacaataacgcggcggccacggattgcccgccattggaattgtttaatagtatcatcgaagatggtgacatggtggataccggttttggctgcatggatttcggcacgttacaggccaacaaaagcgacgtgccaattgatatctgcaattctacctgtaagtacccggattatctgaagatggcgtcggaaccgtacggggatagcctgtttttctttctgcgccgcgagcagatgttcgttcgtcatttctttaaccgcgcgggtaaattgggcgaggccgttccggacgatttgtatattaaaggctcggggaataccgcagtgattcagagtagcgcgtttttccctaccccatctggttcgatcgtgaccagcgaatcgcaactgtttaacaagccgtattggctgcagcgtgcgcagggccacaataacggcatctgctggggtaatcaactgtttgttacggtcgtggacaccacgcggagtacgaacatgacgctgtgtaccgaagtgactaaagagggtacctacaagaatgataactttaaagagtatgttcgccatgttgaagagtatgatttgcagtttgtgttccaattgtgcaaaattacgttaacggccgagatcatgacttacattcacaccatggatagcaatattctggaagactggcagttcggcctgacgccgccaccttctgcctcgctgcaggatacctatcgctttgtgaccagccaggcgatcacgtgccaaaaatataaaacctgcaaacaggccggcacctgcccgccagatccgccaaaggaaaaagaggacccgttgaacaaatatacgttttgggaagttaatttgaaggaaaaattttcggccgatctggaccagttcccactgggccgtaaatttctgttacaaagtggtttgaaagccaagccgcgcttgaaacgtagcgcgccaaccacgcgggcgccgtctaccaaacgcaagaaagttaagaaataa

SEQ ID NO: 23 - DNA, 1455 bp, Artificial Sequence
  gene (1)..(1455): Synthetic 58L1 delta Ch4/16dEs nt
atgagcgtgtggagaccctccgaagcaaccgtctatctcccacccgtccccgtcagcaaagtcgtgtcaaccgacgagtacgtcagcaggacctcaatctactactacgctggttccagtcgcttgctcgccgtcggcaacccctacttcagtattaagtccccaaacaacaacaagaaggtgctggtcccaaaagtgagcggcctgcaataccgcgtgttccgcgtcaggctgcccgacccaaacaagttcggcttccccgacaccagcttctacaatcccgacacccagaggctcgtgtgggcctgcgtgggcttggagatcggccgcggccaaccactcggcgtcggcgtgtccggccacccctacctgaacaagttcgacgataccgagacatccaatcgctacccagcccaacccggcagcgacaatcgcgagtgtctgagcatggactacaagcagacccagctgtgcctgatcggctgcaagcccccaaccggtgaacactggggcaagggcgtcgcttgcaacaacaacgccgccgccaccgactgcccacccctcgagttgttcaacagcatcatcgaagacggcgatatggtggacaccggcttcggctgtatggatttcggcaccctccaagccaacaagtccgacgtccccatcgacatctgcaattccacctgtaagtaccccgactacctgaagatggcatccgagccctacggcgacagcctcttcttcttcctccgcagggaacaaatgttcgtccgccatttcttcaaccgcgccggcaagttgggcgaagccgtgcccgacgatttgtacatcaagggcagtggcaacaccgccgtcattcagtcctccgcattcttcccaaccccctccggcagcatcgtcacaagcgagagccagctgttcaacaagccctactggttgcaaagggcccagggccacaataacggcatctgttggggcaaccaactgttcgtcacagtcgtcgacacaaccaggtcaaccaacatgaccctgtgtaccgaggtgaccaaggagggcacctacaagaacgacaacttcaaagagtacgtgaggcacgtcgaggagtacgatctgcaattcgtcttccaattgtgtaagatcaccttgaccgccgaaatcatgacctacatccacaccatggacagtaacatcctcgaagattggcagttcggcctgacccccccacccagcgcatccctgcaagatacctaccgcttcgtcacaagtcaagccatcacctgtcagaagtacaagacctgcaagcaggccggtacctgcccccctgacccacccaaggagaaagaggaccccctgaacaagtacaccttctgggaagtcaatctgaaagagaaattcagcgccgacttggaccaattcccactcggcaggaagttcctgctgcagagcggcttgtaa

SEQ ID NO: 24 - DNA, 1545 bp, Artificial Sequence
  gene (1)..(1545): Synthetic 58L1 delta N4h4/16dE nt
atgcgtccgtcggaagccaccgtttatctgccacctgtgccggtgagcaaagttgtctcgacggatgaatatgtgagtcgcaccagcatttattactatgcaggttcttcgcgtttgttggcggtgggcaatccgtactttagcatcaaatcgcctaacaataacaaaaaggttttagtgccgaaagtgagtggcctgcaatatcgtgtgtttcgcgtccgcctgccggatccaaataagtttgggttccctgacaccagcttttataacccagatacccagcgtctggtttgggcgtgcgtgggtttggaaattggccgcggccaaccgttgggtgtgggtgtttctggccatccttacttgaataaatttgatgacaccgaaacttcgaaccgttatccggcccagccaggcagcgacaatcgggaatgtctgtcgatggattacaaacaaacgcagctgtgcttaattgggtgtaagccgccaaccggtgagcactggggcaaaggcgttgcatgtaacaataacgcggcggccacggattgcccgccattggaattgtttaatagtatcatcgaagatggtgacatggtggataccggttttggctgcatggatttcggcacgttacaggccaacaaaagcgacgtgccaattgatatctgcaattctacctgtaagtacccggattatctgaagatggcgtcggaaccgtacggggatagcctgtttttctttctgcgccgcgagcagatgttcgttcgtcatttctttaaccgcgcgggtaaattgggcgaggccgttccggacgatttgtatattaaaggctcggggaataccgcagtgattcagagtagcgcgtttttccctaccccatctggttcgatcgtgaccagcgaatcgcaactgtttaacaagccgtattggctgcagcgtgcgcagggccacaataacggcatctgctggggtaatcaactgtttgttacggtcgtggacaccacgcggagtacgaacatgacgctgtgtaccgaagtgactaaagagggtacctacaagaatgataactttaaagagtatgttcgccatgttgaagagtatgatttgcagtttgtgttccaattgtgcaaaattacgttaacggccgagatcatgacttacattcacaccatggatagcaatattctggaagactggcagttcggcctgacgccgccaccttctgcctcgctgcaggatacctatcgctttgtgaccagccaggcgatcacgtgccaaaaactgtataaaacctgcaaacaggccggcacctgcccgccagatattatcccgaaagttgaaggtccgccaaaggaaaaagaggacccgttgaacaaatatacgttttgggaagttaatttgaaggaaaaattttcggccgatctggaccagttcccactgggccgtaaatttctgttacaaagtggtttgaaagccaagccgcgcttgaaacgtagcgcgccaaccacgcgggcgccgtctaccaaacgcaagaaagttaagaaataa

SEQ ID NO: 25 - DNA, 1470 bp, Artificial Sequence
  gene (1)..(1470): Synthetic 58L1 delta N4Ch4/16dE nt
atgagaccctccgaagcaaccgtctatctcccacccgtccccgtcagcaaagtcgtgtcaaccgacgagtacgtcagcaggacctcaatctactactacgctggttccagtcgcttgctcgccgtcggcaacccctacttcagtattaagtccccaaacaacaacaagaaggtgctggtcccaaaagtgagcggcctgcaataccgcgtgttccgcgtcaggctgcccgacccaaacaagttcggcttccccgacaccagcttctacaatcccgacacccagaggctcgtgtgggcctgcgtgggcttggagatcggccgcggccaaccactcggcgtcggcgtgtccggccacccctacctgaacaagttcgacgataccgagacatccaatcgctacccagcccaacccggcagcgacaatcgcgagtgtctgagcatggactacaagcagacccagctgtgcctgatcggctgcaagcccccaaccggtgaacactggggcaagggcgtcgcttgcaacaacaacgccgccgccaccgactgcccacccctcgagttgttcaacagcatcatcgaagacggcgatatggtggacaccggcttcggctgtatggatttcggcaccctccaagccaacaagtccgacgtccccatcgacatctgcaattccacctgtaagtaccccgactacctgaagatggcatccgagccctacggcgacagcctcttcttcttcctccgcagggaacaaatgttcgtccgccatttcttcaaccgcgccggcaagttgggcgaagccgtgcccgacgatttgtacatcaagggcagtggcaacaccgccgtcattcagtcctccgcattcttcccaaccccctccggcagcatcgtcacaagcgagagccagctgttcaacaagccctactggttgcaaagggcccagggccacaataacggcatctgttggggcaaccaactgttcgtcacagtcgtcgacacaaccaggtcaaccaacatgaccctgtgtaccgaggtgaccaaggagggcacctacaagaacgacaacttcaaagagtacgtgaggcacgtcgaggagtacgatctgcaattcgtcttccaattgtgtaagatcaccttgaccgccgaaatcatgacctacatccacaccatggacagtaacatcctcgaagattggcagttcggcctgacccccccacccagcgcatccctgcaagatacctaccgcttcgtcacaagtcaagccatcacctgtcagaagctgtacaagacctgcaagcaggccggtacctgcccccctgacatcatccccaaggtcgaaggaccacccaaggagaaagaggaccccctgaacaagtacaccttctgggaagtcaatctgaaagagaaattcagcgccgacttggaccaattcccactcggcaggaagttcctgctgcagagcggcttgtaa

SEQ ID NO: 26 - DNA, 1527 bp, Artificial Sequence
  gene (1)..(1527): Synthetic 58L1 delta N4h4/16dEs
atgcgtccgtcggaagccaccgtttatctgccacctgtgccggtgagcaaagttgtctcgacggatgaatatgtgagtcgcaccagcatttattactatgcaggttcttcgcgtttgttggcggtgggcaatccgtactttagcatcaaatcgcctaacaataacaaaaaggttttagtgccgaaagtgagtggcctgcaatatcgtgtgtttcgcgtccgcctgccggatccaaataagtttgggttccctgacaccagcttttataacccagatacccagcgtctggtttgggcgtgcgtgggtttggaaattggccgcggccaaccgttgggtgtgggtgtttctggccatccttacttgaataaatttgatgacaccgaaacttcgaaccgttatccggcccagccaggcagcgacaatcgggaatgtctgtcgatggattacaaacaaacgcagctgtgcttaattgggtgtaagccgccaaccggtgagcactggggcaaaggcgttgcatgtaacaataacgcggcggccacggattgcccgccattggaattgtttaatagtatcatcgaagatggtgacatggtggataccggttttggctgcatggatttcggcacgttacaggccaacaaaagcgacgtgccaattgatatctgcaattctacctgtaagtacccggattatctgaagatggcgtcggaaccgtacggggatagcctgtttttctttctgcgccgcgagcagatgttcgttcgtcatttctttaaccgcgcgggtaaattgggcgaggccgttccggacgatttgtatattaaaggctcggggaataccgcagtgattcagagtagcgcgtttttccctaccccatctggttcgatcgtgaccagcgaatcgcaactgtttaacaagccgtattggctgcagcgtgcgcagggccacaataacggcatctgctggggtaatcaactgtttgttacggtcgtggacaccacgcggagtacgaacatgacgctgtgtaccgaagtgactaaagagggtacctacaagaatgataactttaaagagtatgttcgccatgttgaagagtatgatttgcagtttgtgttccaattgtgcaaaattacgttaacggccgagatcatgacttacattcacaccatggatagcaatattctggaagactggcagttcggcctgacgccgccaccttctgcctcgctgcaggatacctatcgctttgtgaccagccaggcgatcacgtgccaaaaactgtataaaacctgcaaacaggccggcacctgcccgccagatattccgccaaaggaaaaagaggacccgttgaacaaatatacgttttgggaagttaatttgaaggaaaaattttcggccgatctggaccagttcccactgggccgtaaatttctgttacaaagtggtttgaaagccaagccgcgcttgaaacgtagcgcgccaaccacgcgggcgccgtctaccaaacgcaagaaagttaagaaataa

SEQ ID NO: 27 - DNA, 1452 bp, Artificial Sequence
  gene (1)..(1452): Synthetic 58L1 delta N4Ch4/16dEs nt
atgagaccctccgaagcaaccgtctatctcccacccgtccccgtcagcaaagtcgtgtcaaccgacgagtacgtcagcaggacctcaatctactactacgctggttccagtcgcttgctcgccgtcggcaacccctacttcagtattaagtccccaaacaacaacaagaaggtgctggtcccaaaagtgagcggcctgcaataccgcgtgttccgcgtcaggctgcccgacccaaacaagttcggcttccccgacaccagcttctacaatcccgacacccagaggctcgtgtgggcctgcgtgggcttggagatcggccgcggccaaccactcggcgtcggcgtgtccggccacccctacctgaacaagttcgacgataccgagacatccaatcgctacccagcccaacccggcagcgacaatcgcgagtgtctgagcatggactacaagcagacccagctgtgcctgatcggctgcaagcccccaaccggtgaacactggggcaagggcgtcgcttgcaacaacaacgccgccgccaccgactgcccacccctcgagttgttcaacagcatcatcgaagacggcgatatggtggacaccggcttcggctgtatggatttcggcaccctccaagccaacaagtccgacgtccccatcgacatctgcaattccacctgtaagtaccccgactacctgaagatggcatccgagccctacggcgacagcctcttcttcttcctccgcagggaacaaatgttcgtccgccatttcttcaaccgcgccggcaagttgggcgaagccgtgcccgacgatttgtacatcaagggcagtggcaacaccgccgtcattcagtcctccgcattcttcccaaccccctccggcagcatcgtcacaagcgagagccagctgttcaacaagccctactggttgcaaagggcccagggccacaataacggcatctgttggggcaaccaactgttcgtcacagtcgtcgacacaaccaggtcaaccaacatgaccctgtgtaccgaggtgaccaaggagggcacctacaagaacgacaacttcaaagagtacgtgaggcacgtcgaggagtacgatctgcaattcgtcttccaattgtgtaagatcaccttgaccgccgaaatcatgacctacatccacaccatggacagtaacatcctcgaagattggcagttcggcctgacccccccacccagcgcatccctgcaagatacctaccgcttcgtcacaagtcaagccatcacctgtcagaagctgtacaagacttgcaagcaggctggtacctgcccccctgacatcccacccaaggagaaagaggaccccctgaacaagtacaccttctgggaagtcaatctgaaagagaaattcagcgccgacttggaccaattcccactcggcaggaagttcctgctgcagagcggcttgtaa

SEQ ID NO: 28 - DNA, 1545 bp, Artificial Sequence
  gene (1)..(1545): Synthetic 58L1 delta N4h4/16dE-CS1 nt
atgagaccctccgaagcaaccgtctatctcccacccgtccccgtcagcaaagtcgtgtcaaccgacgagtacgtcagcaggacctcaatctactactacgctggttccagtcgcttgctcgccgtcggcaacccctacttcagtattaagtccccaaacaacaacaagaaggtgctggtcccaaaagtgagcggcctgcaataccgcgtgttccgcgtcaggctgcccgacccaaacaagttcggcttccccgacaccagcttctacaatcccgacacccagaggctcgtgtgggcctgcgtgggcttggagatcggccgcggccaaccactcggcgtcggcgtgtccggccacccctacctgaacaagttcgacgataccgagacatccaatcgctacccagcccaacccggcagcgacaatcgcgagtgtctgagcatggactacaagcagacccagctgtgcctgatcggctgcaagcccccaaccggtgaacactggggcaagggcgtcgcttgcaacaacaacgccgccgccaccgactgcccacccctcgagttgttcaacagcatcatcgaagacggcgatatggtggacaccggcttcggctgtatggatttcggcaccctccaagccaacaagtccgacgtccccatcgacatctgcaattccacctgtaagtaccccgactacctgaagatggcatccgagccctacggcgacagcctcttcttcttcctccgcagggaacaaatgttcgtccgccatttcttcaaccgcgccggcaagttgggcgaagccgtgcccgacgatttgtacatcaagggcagtggcaacaccgccgtcattcagtcctccgcattcttcccaaccccctccggcagcatcgtcacaagcgagagccagctgttcaacaagccctactggttgcaaagggcccagggccacaataacggcatctgttggggcaaccaactgttcgtcacagtcgtcgacacaaccaggtcaaccaacatgaccctgtgtaccgaggtgaccaaggagggcacctacaagaacgacaacttcaaagagtacgtgaggcacgtcgaggagtacgatctgcaattcgtcttccaattgtgtaagatcaccttgaccgccgaaatcatgacctacatccacaccatggacagtaacatcctcgaagattggcagttcggcctgacccccccacccagcgcatccctgcaagatacctaccgcttcgtcacaagtcaagccatcacctgtcagaagctgtacaagacctgcaagcaggccggtacctgcccccctgacatcatccccaaggtcgaaggaccacccaaggagaaagaggaccccctgaacaagtacaccttctgggaagtcaatctgaaagagaaattcagcgccgacttggaccaattcccactcggcaggaagttcctgctgcagagcggcttgaaagccggcccttcgttggccggctcggcccctacgacctcggcgccgtcgacgggtggcagcgccgtgggtagctaa

SEQ ID NO: 29 - DNA, 1545 bp, Artificial Sequence
  gene (1)..(1545): Synthetic 58L1 delta N4h4/16dE-CS2 nt
atgagaccctccgaagcaaccgtctatctcccacccgtccccgtcagcaaagtcgtgtcaaccgacgagtacgtcagcaggacctcaatctactactacgctggttccagtcgcttgctcgccgtcggcaacccctacttcagtattaagtccccaaacaacaacaagaaggtgctggtcccaaaagtgagcggcctgcaataccgcgtgttccgcgtcaggctgcccgacccaaacaagttcggcttccccgacaccagcttctacaatcccgacacccagaggctcgtgtgggcctgcgtgggcttggagatcggccgcggccaaccactcggcgtcggcgtgtccggccacccctacctgaacaagttcgacgataccgagacatccaatcgctacccagcccaacccggcagcgacaatcgcgagtgtctgagcatggactacaagcagacccagctgtgcctgatcggctgcaagcccccaaccggtgaacactggggcaagggcgtcgcttgcaacaacaacgccgccgccaccgactgcccacccctcgagttgttcaacagcatcatcgaagacggcgatatggtggacaccggcttcggctgtatggatttcggcaccctccaagccaacaagtccgacgtccccatcgacatctgcaattccacctgtaagtaccccgactacctgaagatggcatccgagccctacggcgacagcctcttcttcttcctccgcagggaacaaatgttcgtccgccatttcttcaaccgcgccggcaagttgggcgaagccgtgcccgacgatttgtacatcaagggcagtggcaacaccgccgtcattcagtcctccgcattcttcccaaccccctccggcagcatcgtcacaagcgagagccagctgttcaacaagccctactggttgcaaagggcccagggccacaataacggcatctgttggggcaaccaactgttcgtcacagtcgtcgacacaaccaggtcaaccaacatgaccctgtgtaccgaggtgaccaaggagggcacctacaagaacgacaacttcaaagagtacgtgaggcacgtcgaggagtacgatctgcaattcgtcttccaattgtgtaagatcaccttgaccgccgaaatcatgacctacatccacaccatggacagtaacatcctcgaagattggcagttcggcctgacccccccacccagcgcatccctgcaagatacctaccgcttcgtcacaagtcaagccatcacctgtcagaagctgtacaagacctgcaagcaggccggtacctgcccccctgacatcatccccaaggtcgaaggaccacccaaggagaaagaggaccccctgaacaagtacaccttctgggaagtcaatctgaaagagaaattcagcgccgacttggaccaattcccactcggcaggaagttcctgctgcagagcggcttgggtgccggcccttcgttggccggctcggcccctacgacctcggcgccgtcgacgggtggcagcgccgtgggtagctaa

SEQ ID NO: 30 - DNA, 1545 bp, Artificial Sequence
  gene (1)..(1545): Synthetic 58L1 delta N4h4/16dE-CS3 nt
atgagaccctccgaagcaaccgtctatctcccacccgtccccgtcagcaaagtcgtgtcaaccgacgagtacgtcagcaggacctcaatctactactacgctggttccagtcgcttgctcgccgtcggcaacccctacttcagtattaagtccccaaacaacaacaagaaggtgctggtcccaaaagtgagcggcctgcaataccgcgtgttccgcgtcaggctgcccgacccaaacaagttcggcttccccgacaccagcttctacaatcccgacacccagaggctcgtgtgggcctgcgtgggcttggagatcggccgcggccaaccactcggcgtcggcgtgtccggccacccctacctgaacaagttcgacgataccgagacatccaatcgctacccagcccaacccggcagcgacaatcgcgagtgtctgagcatggactacaagcagacccagctgtgcctgatcggctgcaagcccccaaccggtgaacactggggcaagggcgtcgcttgcaacaacaacgccgccgccaccgactgcccacccctcgagttgttcaacagcatcatcgaagacggcgatatggtggacaccggcttcggctgtatggatttcggcaccctccaagccaacaagtccgacgtccccatcgacatctgcaattccacctgtaagtaccccgactacctgaagatggcatccgagccctacggcgacagcctcttcttcttcctccgcagggaacaaatgttcgtccgccatttcttcaaccgcgccggcaagttgggcgaagccgtgcccgacgatttgtacatcaagggcagtggcaacaccgccgtcattcagtcctccgcattcttcccaaccccctccggcagcatcgtcacaagcgagagccagctgttcaacaagccctactggttgcaaagggcccagggccacaataacggcatctgttggggcaaccaactgttcgtcacagtcgtcgacacaaccaggtcaaccaacatgaccctgtgtaccgaggtgaccaaggagggcacctacaagaacgacaacttcaaagagtacgtgaggcacgtcgaggagtacgatctgcaattcgtcttccaattgtgtaagatcaccttgaccgccgaaatcatgacctacatccacaccatggacagtaacatcctcgaagattggcagttcggcctgacccccccacccagcgcatccctgcaagatacctaccgcttcgtcacaagtcaagccatcacctgtcagaagctgtacaagacctgcaagcaggccggtacctgcccccctgacatcatccccaaggtcgaaggaccacccaaggagaaagaggaccccctgaacaagtacaccttctgggaagtcaatctgaaagagaaattcagcgccgacttggaccaattcccactcggcaggaagttcctgctgcagagcggcttgaaagccggcccttcgttggccggctcggcccctacgaccagagcgccgtcgacgggtggcagcgccgtgggtagctaa

SEQ ID NO: 31 - DNA, 1521 bp, Artificial Sequence
  gene (1)..(1521): Synthetic 58L1h4/16dE
atgagcgtctggcgcccttctgaagcgactgtttacctgccgcccgtcccagtaagcaaggttgtgagcaccgatgaatatgttagtcgtacatcgatctattactatgctggaagctcacgtttgctggcggtaggtaacccttatttttccattaaaagtcccaataataataagaaagttttagtcccgaaggtgtctgggttgcaataccgcgtattccgtgtccgtcttccggacccgaataaatttggcttccccgatacttctttttataatcccgatactcagcgtttagtatgggcctgtgttgggcttgagatcggacgcgggcaacctctgggggttggcgtcagcggccatccttatcttaataaatttgacgatacggagaccagtaaccgctacccggctcagccggggtccgacaaccgtgaatgtttgtccatggattataagcagactcagctgtgtttgatcggttgtaaacctcctacaggtgaacattggggcaaaggtgtagcctgcaataataacgcagcggctaccgattgtccgcctttggagttgttcaactcgattatcgaagacggtgatatggtagacactgggttcggttgcatggactttgggacccttcaagccaataagtccgacgtgccaatcgacatttgcaactctacatgcaagtaccctgactatcttaagatggcgtccgaaccgtacggagattctttgttttttttcctgcgtcgtgagcaaatgttcgtacgccatttcttcaaccgtgctggaaagttaggagaagctgttcctgatgatctgtatattaagggatctgggaacacagcggttattcaatcctccgctttctttccaacgccaagcgggtccattgtgaccagtgaaagccaattatttaacaagccgtactggttacaacgtgcgcagggacacaataatggaatctgctggggcaatcaattgttcgtgacggttgttgacacgacccgttcgaccaatatgaccctttgcactgaggtaactaaggagggtacgtacaaaaatgacaattttaaggagtacgtccgtcatgtcgaggagtatgatcttcagtttgtcttccagttatgtaaaattaccttgaccgcggaaatcatgacgtatatccacacgatggactcaaacatcctggaggattggcaattcggtttgactccgcccccgagcgcgtctcaattgtataagacttgtaagcaggcaggtacatgcccaccagacatcattccaaaagttgaaggaacgtgccaaaagacggcaccaccgaaagaaaaggaggaccccctgaacaaatatacgttttgggaggtgaatttgaaggagaaattctcagcagacttagaccagtttccgcttggccgcaagtttctgttgcagtctgggttgaaggcaaaaccgcgtcttaagcgtagcgcccccacgactcgcgcccccagtacaaaacgtaagaaggttaaaaag

SEQ ID NO: 32 - DNA, 1449 bp, Artificial Sequence
  gene (1)..(1449): Synthetic 58L1 delta Ch4/16dE nt
atgagcgtgtggagaccctccgaagcaaccgtctatctcccacccgtccccgtcagcaaagtcgtgtcaaccgacgagtacgtcagcaggacctcaatctactactacgctggttccagtcgcttgctcgccgtcggcaacccctacttcagtattaagtccccaaacaacaacaagaaggtgctggtcccaaaagtgagcggcctgcaataccgcgtgttccgcgtcaggctgcccgacccaaacaagttcggcttccccgacaccagcttctacaatcccgacacccagaggctcgtgtgggcctgcgtgggcttggagatcggccgcggccaaccactcggcgtcggcgtgtccggccacccctacctgaacaagttcgacgataccgagacatccaatcgctacccagcccaacccggcagcgacaatcgcgagtgtctgagcatggactacaagcagacccagctgtgcctgatcggctgcaagcccccaaccggtgaacactggggcaagggcgtcgcttgcaacaacaacgccgccgccaccgactgcccacccctcgagttgttcaacagcatcatcgaagacggcgatatggtggacaccggcttcggctgtatggatttcggcaccctccaagccaacaagtccgacgtccccatcgacatctgcaattccacctgtaagtaccccgactacctgaagatggcatccgagccctacggcgacagcctcttcttcttcctccgcagggaacaaatgttcgtccgccatttcttcaaccgcgccggcaagttgggcgaagccgtgcccgacgatttgtacatcaagggcagtggcaacaccgccgtcattcagtcctccgcattcttcccaaccccctccggcagcatcgtcacaagcgagagccagctgttcaacaagccctactggttgcaaagggcccagggccacaataacggcatctgttggggcaaccaactgttcgtcacagtcgtcgacacaaccaggtcaaccaacatgaccctgtgtaccgaggtgaccaaggagggcacctacaagaacgacaacttcaaagagtacgtgaggcacgtcgaggagtacgatctgcaattcgtcttccaattgtgtaagatcaccttgaccgccgaaatcatgacctacatccacaccatggacagtaacatcctcgaagattggcagttcggcctgacccccccacctagcgcatcccaactgtacaagacctgcaagcaggccggtacctgcccccctgacatcatccccaaggtcgaaggcacctgtcagaagactgctccacccaaggagaaagaggaccccctgaacaagtacaccttctgggaagtcaatctgaaagagaaattcagcgccgacttggaccaattcccactcggcaggaagttcctgctgcagagcggcttgtaa